(19) United States
(12) Patent Application Publication
(10) Pub. No.: US 2003/0172404 A1
(43) Pub. Date: Sep. 11, 2003

(54) Method of modifying plant characters by the targeted expression of a cell cycle control protein

(76) Inventors: Peter Crook Lloyd John, Farrer (AU); Kerong Zhang, Palmerston (AU); Francis John Sek, Monash (AU); Wim Van Camp, Sint-Denys-Westrem (BE)

Correspondence Address: GREENLEE WINNER AND SULLIVAN P C, 5370 MANHATTAN CIRCLE, SUITE 201, BOULDER, CO, 80303, US

(21) Appl. No.: 10/122,085

(22) Filed: Apr. 10, 2002

Related U.S. Application Data
(63) Continuation-in-part of application No. 09/513,504, filed on Feb. 25, 2000, now abandoned.
(60) Provisional application No. 60/121,870, filed on Feb. 26, 1999.
(60) Provisional application No. 60/149,049, filed on Aug. 16, 1999.

(51) Int. Cl. A01H 5/00

(52) U.S. Cl. 800/287; 800/317.3

(57) ABSTRACT

This invention provides a method for modifying plant growth and development comprising expressing an isolated nucleic acid sequence encoding Cdc25 or a homologue, analogue or derivative thereof in one or more plant cells, tissues or organs operably under the control of a regulatable promoter that does not confer expression throughout the plant under all conditions.

### CROSS REFERENCE TO RELATED APPLICATIONS

This application is a continuation-in-part application of U.S. Ser. N0. 09/513,504 filed on Feb. 25, 2000, which claims the benefit of priority under Title 35 U.S.C. 119(e) from U.S. patent application Ser. No. 60/121,870 filed on Feb. 26, 1999 and U.S. patent application Ser. No. 60/149,049 filed on Aug. 16, 1999. These applications are incorporated herein by reference in their entirety and for all purposes.

### BACKGROUND OF THE INVENTION

The present invention relates generally to a method of modifying plant morphological, biochemical and physiological properties or characteristics, such as one or more environmental adaptive responses and/or developmental processes, said method comprising expressing a cell cycle control protein, in particular Cdc25 phosphoprotein phosphatase, in the plant, operably under the control of a regulatable promoter sequence. Preferably, the characteristics modified by the present invention are cytokinin-mediated and/or gibberellin-mediated characteristics. The present invention extends to gene constructs which are useful for performing the inventive method and to transgenic plants produced therewith having altered morphological and/or biochemical and/or physiological properties compared to their otherwise isogenic counterparts.

This specification contains nucleotide and amino acid sequence information prepared using Patentln Version 3.1, presented herein after the claims. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (eg. SEQ ID NO: 1 refers to the sequence in the sequence listing designated as <400>1).

Those skilled in the art will be aware that the invention described herein is subject to variations and modifications other than those specifically described. It is to be understood that the invention described herein includes all such variations and modifications. The invention also includes all such steps, features, compositions and compounds referred to or indicated in this specification, individually or collectively, and any and all combinations of any two or more of said steps or features.

Throughout this specification, unless the context requires otherwise the word “comprise”, and variations such as “comprises” and “comprising”, will be understood to imply the inclusion of a stated integer or step or group of integers or steps but not the exclusion of any other integer or step or group of integers or steps.

Bibliographic details of the publications referred to by author in this specification are collected at the end of the description.

As used herein, the term “derived from” shall be taken to indicate that a particular integer or group of integers has originated from the species specified, but has not necessarily been obtained directly from the specified source.

Development and environmental adaptation are highly regulated processes in plants. These processes are not cell-autonomous but rather involve extensive communication between different parts of the plant. Amongst the most important mobile signals involved in this long-distance communication are plant hormones such as auxins, cytokinins, abscisic acid, gibberellins, and ethylene. Other signals, so far not defined as plant hormones, include salicyclic acid, jasmonic acid and brassinosteroids.

There are plethora of data showing that the external application of plant hormones has profound effects on development, metabolism and environmental fitness. For example, the external application of cytokinins produces a variety of morphological, biochemical and physiological effects in plants, including the stimulation of organogenesis, shoot initiation from callus cultures, release of lateral buds from apical dominance, dwarf growth, alteration of source/sink relationships, stimulation of pigment synthesis, inhibition of root growth, and delay of senescence. Additionally, exogenous cytokinin application following anthesis in cereals enhances grain set and yield and the phase of nuclear and cell division in the developing endosperm of cereal grains is accompanied by a peak of cytokinin concentration, suggesting a role for cytokinins in grain development in cereals (Herzog, 1980; Morgan et al., 1983). Cytokinins have also been implicated in promoting the initiation of tuber formation in potato (International Patent Publication No. WO 93/07272) and in improving the resistance of potato plants to insects (U.S. Pat. No. 5,496,732) and in inducing male sterility and partial female sterility in tobacco plants (European Patent No. EP-A-334,383).

The effect of cytokinin on plant development and morphology may be attributed, at least in part, to modified biochemistry of the plant, such as a modification to the source/sink relationship in the plant or plant part.

Attempts to modify plant cytokinin-mediated and/or gibberellin-mediated growth and developmental responses employ the exogenous application of cytokinins and/or gibberellins respectively. Such approaches are costly and produce undesirable pleiotropic side-effects on the plant tissue.

Other approaches to modifying plant cytokinin-mediated growth and developmental responses employ the ectopic expression of an introduced bacterial isopentenyladenosine transferase (IPT) gene (International Patent Publication No. WO 93/07272; U.S. Pat. No. 5,496,732; U.S. Pat. No. 5,689,042) under the control of a strong constitutive promoter sequence, developmentally-regulated promoter sequence or hormonally-inducible promoter sequence. Alternatively, plant cytokinin-mediated growth and developmental responses have been modified by the ectopic expression of the Agrobacterium rhizogenes RoIC gene (European Patent No. EP-A-334,383).

Previously, it had been shown that constitutive expression of yeast Cdc25 in tobacco resulted in precocious flowering, more flowers per flowering head, and the presence of “petalless” flowers alongside normal ones, in addition to pleitotropic developmental changes, including altered positioning of the leaves (International Patent Publication No. WO 92/09685; Patent WO 93/12239).

Modified plant growth or architecture has been shown to be effected by modifying expression of the G1 cyclin D3 (International Patent Publication No. WO98/42851). G1 cyclins are required for the induction DNA replication (S phase), and the induction of G1 cyclin expression is dependent on mitogenic signals such as, for example, cytokinin and sucrose (Riou-Khamlichi et al. 1999, Soni et al. 1995). As with the constitutive expression of Cdc25 in plants, prior art methods for modifying plant growth and architecture using these gene include pleiotropic effects.

### SUMMARY OF THE INVENTION

In work leading to the present invention, the present invention determined the significance of Cdc25 in the induction of DNA replication (S phase) under growth-limiting conditions.

Based upon this determination, the present inventors sought to develop a method of producing specific targeted modifications to plant morphology, biochemistry and physiology, in particular specific target modifications to cytokinin-mediated and gibberellin-mediated plant growth and development or aspects thereof, thereby avoiding the problem of pleiotropy associated with the prior art.

Surprisingly, the inventors discovered that the targeted ectopic expression of a cell cycle control protein in particular cells, tissues or organs of the plant would produce localised specific modifications to plant morphology, biochemistry and physiology, compared to otherwise isogenic non-transformed plants.

More particularly, the inventors have discovered that the cytokinin-mediated or gibberellin-mediated induction of mitosis (M phase) in plants, or plant cells, can be obtained by the expression of the yeast Cdc25 phosphoprotein phosphatase therein. The cytokinin-mediated induction of mitosis by Cdc25 is shown in Example 2.

The inventors have also discovered that expression of the yeast Cdc25 phosphoprotein phosphatase in plants or plant cells can override the arrest of DNA replication (S phase) under growth-limiting conditions. Unexpectedly, the same process partially sustains endoreplication or eudoreduplication.

As used herein, the term “growth limiting conditions” shall be taken to mean any developmental or environmental condition that arrests DNA replication in a cell. Accordingly, the imposition of an external environmental condition that limits growth is not essential to make the invention work, and the term “growth limiting conditions” is not to be construed to limit the invention to such an environment. Similarly, “growth limiting conditions” is not equivalent to a condition wherein cells are deprived of exogenous cytokinin. In the exemplified embodiment set forth as Example 9, cell cultures that normally arrested at the G1/S phase were able to proceed beyond that point if ectopically expressing Cdc25. In contrast, non-transformed control cells described in the exemplified embodiment ceased dividing because of a developmental signal that prevented cell density in the culture from being enhanced. The cessation of cell division (i.e., the arrest in G2 phase) was not a consequence of removing cytokinin from the media. Nor was it a consequence of using some sort of sub-optimal or minimal growth media.

Whilst not being bound by any theory or mode of action, it is likely that the ectopic expression of yeast Cdc25 phosphatase in plants releases the inhibition of Cdc2 activity, which is a key enzyme in the control of the cell cycle, and as a consequence, causes isolated cells to enter mitosis and/or S-phase DNA replication. The Cdc25 phosphatase is an intracellular protein, which, unlike exogenously-applied cytokinins or cytokinins produced by ectopic expression of ipt or roIC genes, will only exert a localised effect at the site of protein synthesis. This observation has led the present inventors to develop methods for controlled expression of yeast Cdc25 in particular cells, tissues and organs of plants, for the purposes of mimicking and/or modifying and/or overriding aspects of cytokinin-mediated plant morphology and/or biochemistry and/or physiology, and to facilitate the selection of specific cells, tissues and organs which exhibit cytokinin-mediated morphological characteristics and/or biochemical characteristics and/or physiological characteristics.

Accordingly, one aspect of the invention provides a method of modifying plant morphology and/or biochemistry and/or physiology comprising expressing in particular cells, tissues or organs of a plant, a genetic sequence encoding a cell cycle control protein operably under the control of a regulatable promoter sequence selected from the list comprising cell-specific promoter sequences, tissue-specific promoter sequences, organ-specific promoter sequences, and cell cycle specific gene promoters.

In a particularly preferred embodiment of the invention, the cell cycle control protein is the yeast Cdc25 phosphoprotein phosphatase or a biologically-active homologue, analogue or derivative thereof. The present invention clearly contemplates the use of functional homologues of the fission yeast Cdc25 protein, based upon the evidence provided herein for the presence of Cdc25-like activity and Cdc25-like protein in plants, in particular tobacco (Example 3). Accordingly, the present invention is not limited in application to the use of nucleotide sequences encoding the fission yeast Cdc25 protein, and clearly encompasses the use of Cdc25 polypeptides and genes of plants, preferably monocotyledonous and/or dicotyledonous plants, and more preferably the Cdc25 of a plant as listed or referred to herein.

The ectopic expression of a Cdc25 protein or a homologue, analogue or derivative thereof, can produce a range of desirable phenotypes in plants, such as, for example, by modifying one or more morphological, biochemical, or physiological characteristics as follows: (i) modification of the initiation, promotion, stimulation or enhancement of cell division; (ii) modification of the initiation, promotion, stimulation or enhancement of DNA replication;(iii) modification of the initiation, promotion, stimulation or enhancement of seed set and/or size and/or development; (iv) modification of the initiation, promotion, stimulation or enhancement of tuber formation; (v) modification of the initiation, promotion, stimulation or enhancement of shoot initiation and/or development; (vi) modification of the initiation, promotion, stimulation or enhancement of root initiation and/or development; (vii) modification of the initiation, promotion, stimulation or enhancement of lateral root initiation and/or development; (viii) modification of the initiation, promotion, stimulation or enhancement of nodule formation and/or nodule function; (ix) modification of the initiation, promotion, stimulation or enhancement of bushiness of the plant; (x) modification of the initiation, promotion, stimulation or enhancement of dwarfism in the plant; (xi) modification of the initiation, promotion, stimulation or enhancement of pigment synthesis; (xii) modification of source/sink relationships; (xii) modification of the initiation, promotion, stimulation or enhancement of senescence; and (xiv) modification of stem thickness and/or strength characteristics and/or wind-resistance of the stem.

As used herein, unless specifically stated otherwise, the term “modification of the initiation, promotion, stimulation or enhancement” in relation to a specified integer shall be taken as a clear indication that the integer is capable of being enhanced, increased, stimulated, or promoted, or alternatively, decreased, delayed, repressed, or inhibited.

Preferred embodiments of the invention relate to the effect(s) of cytokinins and/or gibberellins on plant morphology and architecture.

With respect to cytokinins, the present invention clearly contemplates the broad application of the inventive method to the modification of a range of cellular processes, including but not limited to the modification of the initiation, promotion, stimulation or enhancement of cell division and/or seed development and/or tuber formation and/or shoot initiation and/or bushiness and/or dwarfism and/or pigment synthesis, and/or the modification of source/sink relationships, and/or the modification of root growth and/or the inhibition of apical dominance and/or the delay of senescence.

With respect to gibberellins, the present invention clearly contemplates the broad application of the inventive method to the modification of a range of cellular processes, including but not limited to the modification of the initiation, promotion, stimulation or enhancement of cell division and/or seed development and/or tuber formation and/or shoot initiation and/or bushiness and/or dwarfism and/or pigment synthesis, and/or the modification of source/sink relationships, and/or the modification of root growth and/or the inhibition of apical dominance and/or the delay of senescence.

In one preferred embodiment of the present invention, the yeast Cdc25 protein or a homologue, analogue or derivative thereof is expressed operably under the control of a promoter derived from a stem-expressible gene, to increase the strength and thickness of a plant stem to confer improved stability and wind-resistance on the plant. In another preferred embodiment of the present invention, the yeast Cdc25 protein or a homologue, analogue or derivative thereof is expressed in a tuber-forming plant operably under the control of a promoter derived from a stem-expressible gene or tuber-expressible gene, to increase tuber production in the plant.

In another embodiment of the present invention, the yeast Cdc25 protein or a homologue, analogue or derivative thereof is expressed in a tree crop plant such as, but not limited to, Eucalyptus spp. or Populus spp., operably under the control of a promoter derived from a gene that is expressed in vascular tissue and/or cambium cells, to increase lignin content therein. Without being bound by any theory or mode of action, the ectopic expression of Cdc25 under control of a promoter that is operable in vascular tissue and preferably, in cambial cells, will produce thick-stemmed plants and a higher ratio of vascular tissue-to-pith cells within the stem, thereby resulting in more lignin production. Within the vascular tissue, cambial cells contain the highest levels of auxins and are therefore the preferential tissue for Cdc25 overproduction.

In yet another preferred embodiment of the present invention, the yeast Cdc25 protein or a homologue, analogue or derivative thereof is expressed operably under the control of a promoter derived from a seed-expressible gene, to increase seed production in plants, in particular to increase seed set, seed size and seed yield, such as, for example, by enhancing endoreduplication in the seed. More preferably, the promoter is operable in the endosperm of the seed, in which case the combination of the cell cycle-control protein and endosperm-expressible promoter provides the additional advantage of increasing the grain size and grain yield of the plant.

In yet another preferred embodiment of the present invention, the yeast Cdc25 protein or a homologue, analogue or derivative thereof is expressed operably under the control of a promoter derived from a meristem-expressible gene or a shoot-expressible gene or a root-expressible gene, to reduce apical dominance and/or to promote bushiness of the plant and/or to increase or enhance the production of lateral roots, and/or alter leaf shape.

In still another preferred embodiment of the present invention, the yeast Cdc25 protein or a homologue, analogue or derivative thereof is expressed operably under the control of a promoter derived from a leaf-expressible gene, to prevent or delay or otherwise reduce leaf chlorosis and/or leaf necrosis.

In still another preferred embodiment of the present invention, the yeast Cdc25 protein or a homologue, analogue or derivative thereof is expressed operably under the control of a promoter derived from a leaf-expressible gene, to alter leaf shape.

In a further preferred embodiment of the present invention, the yeast Cdc25 protein or a homologue analogue or derivative thereof is expressed under the control of a promoter that is operative in meristem tissue of grain crops, to stimulate cell division in the intercalary meristem of the youngest stem internode and produce greater elongation of the stem and/or to generate a more extensive photosynthetic canopy.

A second aspect of the invention provides a gene construct or vector comprising a nucleotide sequence that encodes a Cdc25 protein or a homologue, analogue or derivative thereof operably under the control of a regulatable promoter sequence selected from the group consisting of:

(i) a plant-expressible cell-specific promoter sequence;

(ii) a plant-expressible tissue-specific promoter sequence;

(iii) a plant-expressible organ-specific promoter sequence;

(iv) a plant-expressible inducible promoter sequence;

(v) a plant-expressible constitutive promoter sequence, wherein the nucleotide sequence encoding the Cdc25 protein and the plant-expressible constitutive promoter sequence are integrated into a transposable element; and

(vi) a plant-expressible cell cycle specific gene promoter sequence.

Preferably, the gene construct or vector according to this aspect of the invention is suitable for expression in a plant cell, tissue, organ or whole plant and more preferably, the subject gene construct or vector is suitable for introduction into and maintenance in a plant cell, tissue, organ or whole plant.

A third aspect of the invention provides a plant cell, tissue, organ or whole plant that has been transformed or transfected with an isolated nucleic acid molecule that comprises a nucleotide sequence which encodes a cell cycle control protein, wherein the expression of said nucleotide sequence is placed operably under the control of a plant-expressible cell-specific promoter sequence, plant-expressible tissue-specific promoter sequence, a plant-expressible organ-specific promoter sequence, plant-expressible cell cycle specific gene promoter or a plant-expressible constitutive promoter sequence such that said plant-expressible constitutive promoter sequence and said nucleotide sequence encoding a cell cycle control protein are integrated into a transposable genetic element.

### DESCRIPTION OF THE DRAWINGS

FIG. 1a is a copy of a photographic representation of a northern blot hybridisation showing the induction of Cdc25 mRNA in tobacco cells containing a dexamethasone-inducible Cdc25 gene, in the absence of exogenous cytokinin. Prior to induction, cells were brought to arrest at the cytokinin control point in late G2 phase by culture without hormone and then with auxin only. Total RNA was extracted from tobacco cells either in the absence of added dexamethasone (lane 0), or after 12 h induction with 0.01 M, or 0.10 μM, or 1.00 μM, or 10.00 μM dexamethasone and then loaded onto agarose gels (60 μg aliquots RNA per lane), transferred to membrane support and probed with a Cdc25-specific probe.

FIG. 1b is a copy of a photographic representation of a western blot showing the induction of p67^{Cdc25} protein in tobacco cells containing a dexamethasone-inducible Cdc25 gene, in the absence of exogenous cytokinin. Prior to induction, cells were brought to arrest at the cytokinin control point in late G2 phase by culture without hormone and then with auxin only. Total protein was extracted from tobacco cells either in the absence of added dexamethasone (lane 1), or after 12 h induction with 0.01 μM dexamethasone (Lane 2), or 0.10 μM dexamethasone (Lane 3), or 1.00 μM dexamethasone (Lane 4), or 10.00 M dexamethasone (Lane 5) and then loaded onto SDS/polyacrylamide gels (50 g aliquots total soluble p67^{cdc25} protein per lane), transferred to membrane support and probed with antibody specific for the Cdc25-specific probe. p67^{Cdc25} was detected by western blot of 50 μg aliquots of total soluble p67^{Cdc25} protein.

FIG. 1c is a copy of a graphical representation showing the induction of cell division in culture, as measured by an increase in cell number, for tobacco cells transformed with a dexamethasone-inducible Cdc25 gene, in the absence of exogenous cytokinin. Prior to induction, cells were brought to arrest at the cytokinin control point in late G2 phase by culture without hormone and then with auxin only. Cell numbers were determined either in the absence of added dexamethasone, or after 12 h induction with 0.01-10.00 μM dexamethasone. Data were also obtained for both transformed cells (◯) and for control non-transformed cells (Δ) grown under identical culture conditions.

FIG. 2a is a copy of a photographic representation showing the activity of Cdc25 phosphatase (Cdc25) and Cdc2 histone kinase (Cdc2) in transgenic tobacco cells containing a dexamethasone-inducible Cdc25 gene and progressing from the late G2 phase hormonal control point into division, that have either not been induced with 0.1 μM dexamethasone (−D), or alternatively, that have been induced with 0.1 μM dexamethasone (+D). The activity of Cdc25 was measured by activation of the tyrosine- phosphorylated Cdc2 enzyme substrate as determined by assaying for phosphorylation of H1 histone by H1 histone kinase. The Cdc25 enzyme from cells induced for 6 hours with dexamethasone was purified using antibodies against authentic fission yeast Cdc25 protein, or alternatively, using preimmune serum (lane marked p-i) or an antibody that had been pre-competed with repeat-freeze-thaw inactivated GST-Cdc25 fusion protein (lane marked p-c). The Cdc2 kinase from cells induced for 12 h with dexamethasone was purified with antibody, or antibody that had been pre-competed with 0.1 mM antigen (lane marked p-c), and assayed by phosphorylation of H1 histone.

FIG. 2b is a graphical representation showing the change in activities of Cdc25 phosphatase (Δ) and Cdc2 histone kinase (◯) in transgenic tobacco cells containing a dexamethasone-inducible Cdc25 gene progressing from the late G2 phase hormonal control point into division and following induction with 0.1 μM dexamethasone. The activities of Cdc25 phosphatase and Cdc2 histone kinase were measured as described for FIG. 2-1.

FIG. 2c is a graphical representation showing the change in cell number (cells/ml×10⁶) of transgenic and non-transgenic tobacco cells containing a dexamethasone-inducible Cdc25 gene, progressing from the late G2 phase hormonal control point into division and following induction with 0.1 μM dexamethasone or cytokinin. Data show cell number for both transgenic cells induced using dexamethasone (□) or cytokinin (Δ), and for non-transgenic cells induced using dexamethasone (◯).

FIG. 2d is a graphical representation showing the change in activities of Cdc25 phosphatase (Δ) and Cdc2 histone kinase (◯) in transgenic tobacco cells containing a dexamethasone-inducible Cdc25 gene, progressing from the late G2 phase hormonal control point into division and following induction with cytokinin in the absence of added dexamethasone. The activities of Cdc25 phosphatase and Cdc2 histone kinase were measured as described for FIG. 2-1.

FIG. 2e is a graphical representation showing the change in activity of Cdc2 histone kinase in transgenic tobacco cells containing a dexamethasone-inducible Cdc25 gene, progressing from the late G2 phase hormonal control point into division and following their stimulation with cytokinin. The Cdc2 histone kinase was purified using p13^{suc1} beads and treated with GST-Cdc25 fusion protein that had been produced in Escherichia coli cells. Data indicate the Cdc2 activity before Cdc25 treatment (◯), and after treatment (●) with cytokinin.

FIG. 2f is a copy of a photographic representation showing the activation of Cdc2 histone kinase by Cdc25 phosphatase in transgenic tobacco cells containing a dexamethasone-inducible Cdc25 gene, prior to stimulation with cytokinin (lanes 1-3) or following 3 hours stimulation with cytokinin (lanes 4-6). Detectable Cdc2 activity was observed in control samples that had been incubated without added Cdc25 (lanes 1 and 4), or following incubation with (i) immunoprecipitated Cdc25 that had been derived from non-transgenic tobacco cells induced with cytokinin for 6 hours (lanes 2 and 5); or (ii) Cdc25 derived from transgenic tobacco cells containing a dexamethasone-inducible Cdc25 gene that had been induced with dexamethasone for 6 hours (lanes 3 and 6). The activity of Cdc2 histone kinase was measured as described for FIG. 2-1. Detection of Cdc25 activity in the immuno-recovered fraction derived from non-transgenic cells indicates the presence of a plant-encoded Cdc25.

FIG. 2g is a copy of a photographic representation showing the presence of phosphorylated tyrosine in Cdc2a (arrow) following induction of transgenic tobacco cells containing a dexamethasone-inducible Cdc25 gene with dexamethasone. The Cdc2a protein was immuno-precipitated with purified antibody, or with antibody precompeted with repeat-freeze-thaw inactivated GST-Cdc25 (lane marked p-c). The upper band indicated in the Figure represents excess IgG.

FIG. 3 is a copy of a photographic representation of a western blot showing purified plant-derived Cdc25 protein. The arrow indicates the plant Cdc25 polypeptide. Anti-GST-Cdc25 antibody at a dilution of 1:500 in buffered saline was used to probe affinity-purified plant Cdc25 protein alone (lane 1) or affinity-purified plant Cdc25 protein following incubation for 1 hour with 0.1 mM GST-Cdc25 fusion protein. Molecular weight markers indicating the molecular mass (kDa) of proteins are indicated at the left of the Figure.

FIG. 4 is a copy of a photographic representation showing the cytokinin-dependent proliferation of tobacco cells in culture. Cell proliferation was detected by the incorporation of BrdU into nuclear DNA of excised tobacco pith tissue primary culture on MS medium either without added hormone (panels a,b), or supplemented with 5.4 μM NAA (panels c,d) or with 0.56 μM BAP (panels e,f) or 5.4 μM NM plus 0.56 μM BAP (panels g,h). Cell cultures shown in panels a, c, e, and g have been stained with DAPI, to detect nuclei. Cell cultures shown in panels b, d, f, and h have been incubated with BrdU, and BrdU-containing DNA has been detected by fluorescence of antibody specific for BrdU-containing DNA.

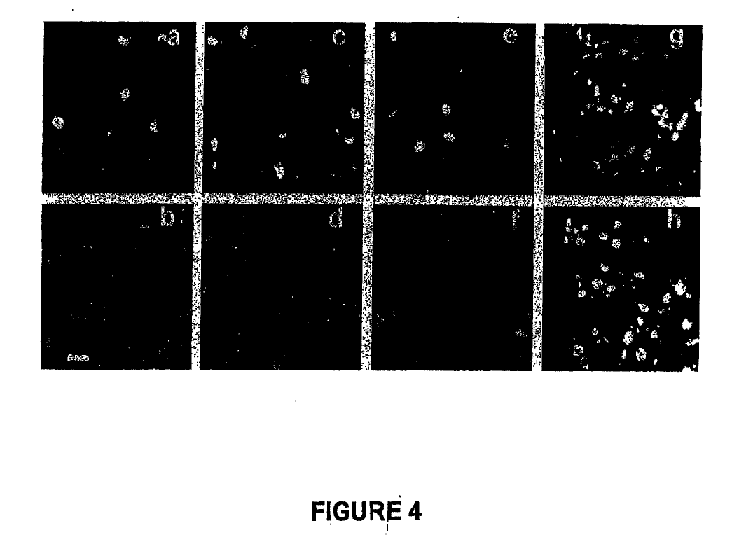

FIG. 5 is a graphical representation showing enhanced cell endoreduplication in tobacco cells expressing Cdc25. Data on the x-axis indicate nuclei having ploidies of 2n, 4n and 8n in cultures in which cell division is arresting. Data on the y-axis indicates the frequency of nuclei for each ploidy. Panel (A) shows nontransgenic cells after 6 days of culture. Panel (B) shows transgenic cells after 6 days, in which the Cdc25 gene is expressed under the control of a glucocorticoid dexamethasone. After 6 days of batch culture, cell cycle progress had been arrested at a G1/S control point in non-transgenic cells (2N peak in A), but has been driven through this point in a majority of the transgenic cells to produce cells that are mostly in G2 phase (4n peak in B). In some cases, an additional traverse of S phase has been induced in transgenic cells, without intervening mitosis, resulting in endoreduplication of the genome and generation of 8n nuclei.

### DETAILED DESCRIPTION OF THE INVENTION

One aspect of the invention provides a method of modifying one or more plant morphological and/or biochemical and/or physiological characteristics comprising expressing in one or more particular cells, tissues or organs of a plant, a cell cycle control protein operably under the control of a regulatable promoter sequence selected from the list comprising cell-specific promoter sequences, tissue-specific promoter sequences, organ-specific promoter sequences, and cell cycle specific gene promoter sequences.

Preferably, the plant morphological, biochemical or physiological characteristic which is modified is a cytokinin-mediated or a gibberellin-mediated characteristic.

The word “modify” or variations such as “modifying” or “modified” as used herein with reference to any specified integer or group of integers shall be taken to indicate that said integer is altered by the performance of one or more steps pertaining to the invention described herein, compared to said integer in the absence of such performance.

Accordingly, by “modifying one or more plant morphological and/or biochemical and/or physiological characteristics” is meant that one or more morphological and/or biochemical and/or physiological characteristics of a plant is altered by the performance of one or more steps pertaining to the invention described herein.

“Plant morphology” or the term “plant morphological characteristic” or similar term will be understood by those skilled in the art to refer to the external appearance of a plant, including any one or more structural features or combination of structural features thereof. Such structural features include the shape, size, number, position, colour, texture, arrangement, and patternation of any cell, tissue or organ or groups of cells, tissues or organs of a plant, including the root, stem, leaf, shoot, petiole, trichome, flower, petal, stigma, style, stamen, pollen, ovule, seed, embryo, endosperm, seed coat, aleurone, fibre, cambium, wood, heartwood, parenchyma, aerenchyma, sieve element, phloem or vascular tissue, amongst others.

As will be known to those skilled in the art, plants may modify one or more plant morphological characteristics in response to the external stimuli, such as, for example, a plant pathogenic infection, or an external stress (drought, flooding, salt stress, dehydration, heavy metal contamination, mineral deficiency, etc). Accordingly, for the present purpose, it shall be understood that a plant morphological characteristic that has been modified in response to one or more external stimuli is within the scope of the inventive method described herein, notwithstanding that the imposition of said external stimuli is not an essential feature of the present invention.

“Plant biochemistry” or the term “plant biochemical characteristic” or similar term will be understood by those skilled in the art to refer to the metabolic and catalytic processes of a plant, including primary and secondary metabolism and the products thereof, including any small molecules, macromolecules or chemical compounds, such as but not limited to starches, sugars, proteins, peptides, enzymes, hormones, growth factors, nucleic acid molecules, celluloses, hemicelluloses, calloses, lectins, fibres, pigments such as anthocyanins, vitamins, minerals, micronutrients, or macronutrients, that are produced by plants.

“Plant physiology” or the term “plant physiological characteristic” or similar term will be understood to refer to the functional processes of a plant, including developmental processes such as growth, expansion and differentiation, sexual development, sexual reproduction, seed set, seed development, grain filling, asexual reproduction, cell division, dormancy, germination, light adaptation, photosynthesis, leaf expansion, fibre production, secondary growth or wood production, amongst others; responses of a plant to externally-applied factors such as metals, chemicals, hormones, growth factors, environment and environmental stress factors (eg. anoxia, hypoxia, high temperature, low temperature, dehydration, light, daylength, flooding, salt, heavy metals, amongst others), including adaptive responses of plants to said externally-applied factors.

Preferably, the ectopic expression of a Cdc25 substrate or modified Cdc25 substrate protein produces a wide range of desirable phenotypes in the plant, selected from the group consisting of:(i) enhanced growth and/or enhanced vigour of the plant; (ii) increased total biomass of the plant; (iii) increased cell number; (iv) reduced flowering time; (v) increased inflorescence formation; (vi) reduced time to seed set (vii) enhanced seed set; (viii) enhanced seed size; (ix) enhanced grain yield; (x) enhanced stem strength; (xi) enhanced stem thickness; (xii) enhanced stem stability; (xiii) enhanced wind-resistance of the stem; (xiv) enhanced tuber formation; (xv) enhanced tuber development; (xvi) increased lignin content; (xvii) enhanced ploidy of the seed; (xviii) enhanced endosperm size; (xix) reduced apical dominance; (xx) increased bushiness; (xxi) enhanced lateral root formation; (xxii) enhanced rate of lateral root production; (xxiii) enhanced nitrogen-fixing capability; (xxiv) enhanced nodulation or nodule size; (xxv) reduced or delayed leaf chlorosis; (xxvi) reduced or delayed leaf necrosis; (xxvii) partial or complete inhibition of the arrest of DNA replication in a plant cell under growth-limiting conditions; (xxviii) enhanced endoreplication; (xxix) enhanced endoreduplication, including enhanced endoreduplication in the seed; and (xxx) enhanced cell expansion.

More preferably, the plant morphological, biochemical or physiological characteristic which is modified is a cytokinin-mediated or a gibberellin-mediated characteristic selected from the group consisting of: (i) enhanced stem thickness; (ii) enhanced stem stability; (iii) enhanced wind-resistance of the stem; (iv) enhanced tuber formation; (v) enhanced tuber development; (vi) increased lignin content; (vii) enhanced seed set; (viii) enhanced seed production; (ix) enhanced grain yield; (x) enhanced ploidy of the seed; (xi) enhanced endosperm size; (xii) reduced apical dominance; (xiii) increased bushiness; (xiv) enhanced lateral root formation; (xv) enhanced rate of lateral root production; (xvi) enhanced nitrogen-fixing capability; (xvii) enhanced nodulation or nodule size; (xviii) reduced or delayed leaf chlorosis; (xix) reduced or delayed leaf necrosis; (xx) partial or complete inhibition of the arrest of DNA replication in a plant cell under growth-limiting conditions; (xxi) enhanced endoreplication and/or enhanced endoreduplication; and (xxii) enhanced cell expansion.

In an alternative embodiment, the plant morphological, biochemical or physiological characteristic which is modified is selected from the group consisting of: (i) plant growth and vigour; (ii) total biomass; (iii) cell number; (iv) flowering time; (v) branching; (vi) inflorescence formation; and (vii) seed set and/or seed yield, and/or seed size. According to this embodiment of the present invention, expression of the cell cycle control protein preferably leads to increased plant productivity such as, for example, increased growth and vigour; increased total biomass; increased cell number; reduced flowering time; increased branching; increased inflorescence formation; and increased seed set; amongst others.

In a particularly preferred embodiment, the modified plant characteristic is other than enhanced lateral root formation or enhanced rate of lateral root formation, particularly as a consequence of merely stimulating the transition from the G2 phase to the M phase in cells of the root pericycle (i.e. the layer of the root internal to the endodermis which are in the G2 phase having already replicated their DNA). In cells that are arrested in the G2 phase, enhanced lateral root formation produced as a consequence of ectopically expressing Cdc25 is not necessarily a consequence of inhibiting the arrest in DNA replication (i.e. the transition from S phase to M phase).

The word “express” or variations such as “expressing” and “expression” as used herein shall be taken in their broadest context to refer to the transcription of a particular genetic sequence to produce sense or antisense mRNA or the translation of a sense mRNA molecule to produce a peptide, polypeptide, oligopeptide, protein or enzyme molecule. In the case of expression comprising the production of a sense mRNA transcript, the word “express” or variations such as “expressing” and “expression” may also be construed to indicate the combination of transcription and translation processes, with or without subsequent post-translational events which modify the biological activity, cellular or sub-cellular localization, turnover or steady-state level of the peptide, polypeptide, oligopeptide, protein or enzyme molecule.

The term “cell cycle” as used herein shall be taken to include the cyclic biochemical and structural events associated with growth and with division of cells, and in particular with the regulation of the replication of DNA and mitosis. Cell cycle includes phases called: GO (gap 0), G1 (gap 1), DNA replication (S), G2 (gap 2), and mitosis including cytokinesis (M). Normally these four phases occur sequentially. However, the cell cycle also includes modified cycles such as endomitosis, acytokinesis, polyploidy, polyteny, endopolyploidisation and endoreduplication or endoreplication.

The term “cell cycle interacting protein”, “cell cycle protein”, or “cell cycle control protein” as denoted herein means a protein which exerts control on or regulates or is required for the cell cycle or part thereof of a cell, tissue, organ or whole organism and/or DNA replication. It may also be capable of binding to, regulating or being regulated by cyclin dependent kinases or their subunits. The term also includes peptides, polypeptides, fragments, variant, homologs, alleles or precursors (eg preproproteins or preproteins) thereof.

Cell cycle control proteins and their role in regulating the cell cycle of eukaryotic organisms are reviewed in detail by John (1981) and the contributing papers therein (Norbury and Nurse 1992); Nurse 1990; Ormong and Francis 1993) and the contributing papers therein (Doerner et al. 1996; Elledge 1996; Francis and Halford 1995; Francis et al. 1998; Hirt et al. 1991; Mironov et al. 1999) which are incorporated herein by way of reference.

The term “cell cycle control gene” refers to any gene or mutant thereof which exerts positive or negative control on, or is required for, chromosomal DNA synthesis, mitosis (preprophase band, nuclear envelope, spindle formation, chromosome condensation, chromosome segregation, formation of new nuclei, formation of phragmoplast, etc) meiosis, cytokinesis, cell growth, or endoreduplication. The term “cell cycle control gene” also includes any and all genes that exert control on a cell cycle protein as hereinbefore defined, including any homologues of CDKs, cyclins, E2Fs, Rb, CKI, Cks, cyclin D, Cdc25, Wee1, Nim1, MAP kinases, etc. Preferably, a cell cycle control gene will exert such regulatory control at the post-translation level, via interactions involving the polypeptide product expressed therefrom.

More specifically, cell cycle control genes are all genes involved in the control of entry and progression through S phase. They include, not exclusively, genes expressing “cell cycle control proteins” such as cyclin dependent kinases (CDK), cyclin dependent kinase inhibitors (CKI), D, E and A cyclins, E2F and DP transcription factors, pocket proteins, CDC7/DBF4 kinase, CDC6, MCM2-7, Orc proteins, Cdc45, components of SCF ubiquitin ligase, PCNA, and DNA-polymerase, amongst others.

The term “cell cycle control protein” includes cyclins A, B, C, D and E, including CYCA1;1, CYCA2;1, CYCA3;1, CYCB1;1, CYCB1;2, CYCB2;2, CYCD1;1, CYCD2;1. CYCD3;1, and CYCD4;1 (Evans et al. 1983; Francis et al. 1998; Labbe et al. 1989; Murray and Kirschner 1989; Renaudin et al. 1996; Soni et al 1995; Sorrell et a/ 1999; Swenson et al 1986); cyclin dependent kinase inhibitor (CKI) proteins such as ICK1 (Wang et al 1997), FL39, FL66, FL67 (PCT/EP98/05895), Sic1, Far1, Rum1, p21, p27, p57, p16, p15, p18, p19 (Elledge 1996; Pines 1995a,b), p14 and p14ARF; p13suc1 or CKS1At (De Veylder et al 1997; Hayles and Nurse 1986) and nim-1 (Russell and Nurse 1987a; Russell and Nurse 1987b; Fantes 1989; Russell and Nurse 1986; Russell and Nurse 1987a; Russell and Nurse 1987b) homologues of Cdc2 such as Cdc2MsB (Hirt et al 1993) CdcMs kinase (Bogre et al 1997) Cdc2 T14Y15 phosphatases such as Cdc25 protein phosphatase or p8OCdc25 (Bell et al 1993; Elledge 1996; Kumaghi and Dunphy 1991; Russell and Nurse 1986) and Pyp3 (Elledge 1996) Cdc2 protein kinase or p34Cdc2 (Colasanti et al 1991; Feiler and Jacobs 1990; Hirt et al 1991; John et al 1989; Lee and Nurse 1987; Nurse and Bissett 1981; Ormrod and Francis 1993) Cdc2a protein kinase (Hemerly et al 1993) Cdc2 T14Y15 kinases such as weel or p107weel (Elledge 1996; Russell and Nurse 1986; Russell and Nurse 1987a; Russell and Nurse 1987a; Sun et al 1999) mik1 (Lundgren et al 1991) and myt1 (Elledge 1996); Cdc2 T161 kinases such as Cak and Civ (Elledge 1996); Cdc2 T161 phosphatases such as Kap1 (Elledge 1996); Cdc28 protein kinase or p34Cdc28 (Nasmyth 1993; Reed et al. 1985) p40MO15 (Fequet et al 1993; Poon et al. 1993) chkl kinase (Zeng et al 1998) cdsl kinase (Zeng et al 1998) growth associated H1 kinase (Labbe et al 1989; Lake and Salzman 1972; Langan 1978; Zeng et al 1998) MAP kinases described by (Binarova et al 1998; Bögre et al 1999; Calderini et al 1998; Wilson et al 1999).

Other cell cycle control proteins are involved in cyclin D-mediated entry of cells into G1 from GO include pRb (Xie et al., 1996; Huntley et al., 1998), E2F, RIP, MCM7, and the pRb-like proteins p107 and p130.

Other cell cycle control proteins are involved in the formation of a pre-replicative complex at one or more origins of replication, such as, but not limited to, ORC, CDC6, CDC14, RPA and MCM proteins or in the regulation of formation of this pre-replicative complex, such as, but not limited to, the CDC7, DBF4 and MBF proteins.

For the present purpose, the term “cell cycle control protein” shall further be taken to include any one or more of those proteins that are involved in the turnover of any other cell cycle control protein, or in regulating the half-life of said other cell cycle control protein. The term “protein turnover” is to include all biochemical modifications of a protein leading to the physical or functional removal of said protein. Although not limited to these, examples of such modifications are phosphorylation, ubiquitination and proteolysis. Particularly preferred proteins which are involved in the proteolysis of one or more of any other of the above-mentioned cell cycle control proteins include the yeast-derived and animal-derived proteins, Skp1, Skp2, Rub1, Cdc20, cullins, CDC23, CDC27, CDC16, and plant-derived homologues thereof (Cohen-Fix and Koshland 1997; Hochstrasser 1998; Krek 1998; Lisztwan et al 1998) and Plesse et al in (Francis et al 1998)).

For the present purpose, the term “cell cycle control genes” shall further be taken to include any one or more of those gene that are involved in the transcriptional regulation of cell cycle control gene expression such as transcription factors and upstream signal proteins. Additional cell cycle control genes are not excluded.

For the present purpose, the term “cell cycle control genes” shall further be taken to include any cell cycle control gene or mutant thereof, which is affected by environmental signals such as for instance stress, nutrients, pathogens, or by intrinsic signals such as the animal mitogens or the plant hormones (auxins, cytokinins, ethylene, gibberellic acid, abscisic acid and brassinosteroids).

In a preferred embodiment, the cell cycle control protein used in performing the invention is a Cdc25 protein or a homologue, analogue, or derivative thereof. In all cells, the switch that raises activity of Cdc2 at entry into mitosis is the Cdc25-catalysed removal of phosphate from theonine-14 and/or tyrosine-15 in Cdc2. Prior to the onset of mitosis the activity of the Cdc2 protein involved in this process is inactivated by the wee-1-mediated and/or mik-1- mediated phosphorylation of threonine-14 and/or tyrosine-15 in Cdc2. In yeasts there is only one CDK (Cdc2), and the Cdc25-catalysed removal of phosphate from tyrosine-15 in Cdc2 occurs only once in the cell cycle, at the G2/M phase transition. In contrast, higher eukaryotic cells contain several CDKs (animal and plant cells) and several Cdc25 proteins (animal cells). In mammals, the molecular switch of Cdc25-catalysed removal of phosphate from threonine-14 and/or tyrosine-15 in Cdc2 is also used at entry into the S phase, and a separate CDK (CDK2) and a separate Cdc25 (Cdc25A) perform this function. In plants, whilst it is known there are several CDKs, it is not known if there is a single CDK that is controlled at S phase, like CDK2 by the status of threonine-14 and/or tyrosine-15 phosphorylation.

The present invention encompasses altering the balance between Cdc25 dephosphorylation and wee-1/mik-1 phosphorylation of their substrates either by overexpression of Cdc25 and/or down-regulation of wee-1/mik-1.

The present invention provides evidence that the G2/M phase transition, and, surprisingly, the G1/S phase transition of plant cells can be modified by the expression of a single Cdc25 in plant cells.

“Homologues” of a Cdc25 protein are those peptides. oligopeptides, polypeptides, proteins and enzymes which contain amino acid substitutions, deletions and/or additions relative to the Cdc25 polypeptide without altering one or more of its cell cycle control properties, in particular without reducing the ability of the Cdc25 polypeptide to induce one or more aspects of cytokinin-mediated and/or gibberellin-mediated effects in a plant cell, tissue, organ or whole organism.

To produce such homologues of a cell cycle control protein such as Cdc25, amino acids present in Cdc25 can be replaced by other amino acids having similar properties, for example hydrophobicity, hydrophilicity, hydrophobic moment, antigenicity, propensity to form or break (x-helical structures or β-sheet structures, and so on.

Substitutional variants are those in which at least one residue in the Cdc25 amino acid sequence has been removed and a different residue inserted in its place. Amino acid substitutions are typically of single residues, but may be clustered depending upon functional constraints placed upon the polypeptide; insertions will usually be of the order of about 1-10 amino acid residues and deletions will range from about 1-20 residues. Preferably, amino acid substitutions will comprise conservative amino acid substitutions, such as those described supra.

Insertional amino acid sequence variants are those in which one or more amino acid residues are introduced into a predetermined site in the Cdc25 protein. Insertions can comprise amino-terminal and/or carboxyl terminal fusions as well as intra-sequence insertions of single or multiple amino acids. Generally, insertions within the amino acid sequence will be smaller than amino or carboxyl terminal fusions, of the order of about 1 to 4 residues.

Deletional variants are characterised by the removal of one or more amino acids from the Cdc25 sequence.

Amino acid variants of the Cdc25 polypeptide may readily be made using peptide synthetic techniques well known in the art, such as solid phase peptide synthesis and the like, or by recombinant DNA manipulations. The manipulation of DNA sequences to produce variant proteins which manifest as substitutional, insertional or deletional variants are well known in the art. For example, techniques for making substitution mutations at predetermined sites in DNA having known sequence are well known to those skilled in the art, such as by M1 3 mutagenesis or other site-directed mutagenesis protocol.

“Analogues” of a Cdc25 protein are defined as those peptides, oligopeptides, polypeptides, proteins and enzymes which are functionally equivalent to the Cdc25 polypeptide in inducing one or more cytokinin-mediated and/or gibberellin-mediated effects in plant cells, tissues, organs or whole organisms.

Preferred analogues of the Cdc25 protein are those peptides, polypeptide, proteins, and enzymes, that function as a substrate of Cdc25 or a modified substrate of Cdc25 in a plant cell and/or tissue and/or organ and/or whole plant.

Preferably, the Cdc25 analogue is other than a Cdc2a protein or a cyclin B protein, or a homologue or derivative thereof, notwithstanding that such proteins are useful in performing the present invention.

In the present context, the term “substrate of Cdc25” shall be taken to refer to any protein that interacts with Cdc25 in regulating the plant cell cycle, including, but not limited to cyclin-dependent kinases (CDKs), the most significant of which is Cdc2, which in all cells is the key enzyme driving entry into mitosis.

Without being bound by any theory or mode of action, the substitution or deletion of the phosphorylation sites of a Cdc2 protein mimics the effect of a constitutive phosphatase activity, such as the effect of the Cdc25 protein phosphatase (p80^{Cdc25}) activity. Moreover, the substitution or deletion of the phosphorylation sites of a Cdc2a protein further mimics the effect of down-regulated kinase activity, such as a down-regulation of the wee-1 kinase and/or mik-1. Those skilled in the art will know that the wee-1 and mik-1 kinase adds the inhibitory phosphate on threonine-14 and/or tyrosine 15. Thus phosphorylated protein will not be produced at high steady state concentrations in either the absence of phosphorylation or when phosphatase(s) is(are) expressed at raised levels, or when kinase(s) is(are) expressed at lowered levels. Accordingly, the modified Cdc2a activity effects described herein can also be obtained, albeit only in part and without the benefits derived from increasing the amount of active Cdc2a protein, by the regulated expression of Cdc25. Alternatively, the modified Cdc2a activity effects described herein can also be obtained by down-regulating, or inhibiting, wee-1 and/or mik-1 kinase activity, such as, for example, by using antisense molecules, ribozymes, cosuppression molecules, gene targeting molecules, or gene silencing molecules, etc., which target the wee-1 and/or mik-1 genes or gene products, respectively.

The term “modified substrate of Cdc25” refers to a homologue, analogue or derivative of a substrate of Cdc25 that mimics the effect of Cdc25 activity, in particular a non-phosphorylatable Cdc25 substrate that mimics the effect of Cdc25 activity or alternatively mimics the effects of down-regulated wee-1 and/or mik-I kinase. For example, substitution of threonine and tyrosine at positions 14 and 15 of cyclin-dependent kinases (CDKs) for alanine and phenylalanine, respectively, can produce one or more cytokinin-like effects in the plant, similar to those observed following constitutive Cdc25 expression in the plant or alternatively to those observed following down-regulation of wee-1 and/or mik-1. Notwithstanding that this may be the case, the effects of CDK(A₁₄F,5 ) expression are inferior to those of naturally-occurring or wild-type Cdc25, possibly because plant cells comprise several Cdc25 substrates.

Accordingly, similar effects to the Cdc25-induced effects obtained by expressing Cdc25 under control of the regulatable promoter, can be obtained by expressing the Cdc25 substrate or a modified form thereof operably under control of the same or a functionally-equivalent promoter. The present invention clearly extends to such arrangements.

The present invention extends further to the co-expression of Cdc25 and one or more Cdc25 substrates and/or one or more modified Cdc25 substrates, operably under the control of a regulatable promoter that is selected for a particular application as described herein.

“Derivatives” of a Cdc25 protein are those peptides, oligopeptides, polypeptides, proteins and enzymes which comprise at least about five contiguous amino acid residues of a naturally-occurring Cdc25 polypeptide, in particular the fission yeast p80 Cdc25 polypeptide, but which retain activity in the induction of one or more cytokinin-mediated and/or gibberellin-mediated effects in a plant cell, tissue, organ or whole organism. A “derivative” may further comprise additional naturally-occurring, altered glycosylated, acylated or non-naturally occurring amino acid residues compared to the amino acid sequence of a naturally-occurring Cdc25 polypeptide. Alternatively or in addition, a derivative may comprise one or more non-amino acid substituents compared to the amino acid sequence of a naturally-occurring Cdc25 polypeptide, for example a reporter molecule or other ligand, covalently or non-covalently bound to the amino acid sequence such as, for example, a reporter molecule which is bound thereto to facilitate its detection.

Other examples of recombinant or synthetic mutants and derivatives of the Cdc25 polypeptide include those incorporating single or multiple substitutions, deletions and/or additions therein, such as carbohydrates, lipids and/or proteins or polypeptides. Naturally-occurring or altered glycosylated or acylated forms of the Cdc25 polypeptide are also contemplated by the present invention. Additionally, homopolymers or heteropolymers comprising one or more copies of the Cdc25 polypeptide are within the scope of the invention, the only requirement being that such molecules possess biological activity in inducing one or more cytokinin-mediated and/or gibberellin-mediated effects in plant cells, tissues, organs or whole organisms.

Preferred homologues, analogues and derivatives of the fission yeast Cdc25 polypeptide contemplated by the present invention are derived from plants. As exemplified herein, the present inventors have identified a Cdc25 activity in tobacco cells which is contemplated as being of particular use in performing the various embodiments described herein.

In a particularly preferred embodiment of the invention, the cell cycle control protein is the yeast Cdc25 phosphoprotein phosphatase or a biologically-active homologue, analogue or derivative thereof and in particular, a plant-derived homologue of the yeast Cdc25 phosphoprotein phosphatase. The present invention clearly contemplates the use of functional homologues of the fission yeast Cdc25 protein, based upon the evidence provided herein for the presence of Cdc25-like activity and Cdc25-like protein in tobacco (Example 3). Accordingly, the present invention is not limited in application to the use of nucleotide sequences encoding the fission yeast p80^{Cdc25} protein.

To effect expression of the cell cycle control protein in a plant cell, tissue or organ, either the protein may be introduced directly to said cell, such as by microinjection means or alternatively, an isolated nucleic acid molecule encoding said protein may be introduced into the cell, tissue or organ in an expressible format.

By “expressible format” is meant that the isolated nucleic acid molecule is in a form suitable for being transcribed into mRNA and/or translated to produce a protein, either constitutively or following induction by an intracellular or extracellular signal, such as an environmental stimulus or stress (anoxia, hypoxia, temperature, salt, light, dehydration, etc) or a chemical compound such as an antibiotic (tetracycline, ampicillin, rifampicin, kanamycin) hormone (eg. gibberellin, auxin, cytokinin, glucocorticoid, etc), hormone analogue (iodoacetic acid (IAA), 2,4-D, etc), metal (zinc, copper, iron, etc), or dexamethasone, amongst others. As will be known to those skilled in the art, expression of a functional protein may also require one or more post-translational modifications, such as glycosylation, phosphorylation, dephosphorylation, or one or more protein-protein interactions, amongst others. All such processes are included within the scope of the term “expressible format”.

Preferably, expression of a cell cycle control protein in a specific plant cell, tissue, or organ is effected by introducing and expressing an isolated nucleic acid molecule encoding said protein, such as a cDNA molecule, genomic gene, synthetic oligonucleotide molecule, mRNA molecule or open reading frame, to said cell, tissue or organ, wherein said nucleic acid molecule is placed operably in connection with a suitable plant-expressible promoter sequence.

Reference herein to a “promoter” is to be taken in its broadest context and includes the transcriptional regulatory sequences derived from a classical eukaryotic genomic gene, including the TATA box which is required for accurate transcription initiation, with or without a CCAAT box sequence and additional regulatory elements (i.e. upstream activating sequences, enhancers and silencers) which alter gene expression in response to developmental and/or external stimuli, or in a tissue-specific manner.

The term “promoter” also includes the transcriptional regulatory sequences of a classical prokaryotic gene, in which case it may include a -35 box sequence and/or a −10 box transcriptional regulatory sequences.

The term “promoter” is also used to describe a synthetic or fusion molecule, or derivative which confers, activates or enhances expression of a nucleic acid molecule in a cell, tissue or organ.

Preferred promoters may contain additional copies of one or more specific regulatory elements, to further enhance expression and/or to alter the spatial expression and/or temporal expression of a nucleic acid molecule to which it is operably connected. For example, copper-responsive, glucocorticoid-responsive or dexamethasone-responsive regulatory elements may be placed adjacent to a heterologous promoter sequence driving expression of a nucleic acid molecule to confer copper inducible, glucocorticoid-inducible, or dexamethasone-inducible expression respectively, on said nucleic acid molecule.

In the context of the present invention, the promoter is a plant-expressible promoter sequence. By “plant-expressible” is meant that the promoter sequence, including any additional regulatory elements added thereto or contained therein, is at least capable of inducing, conferring, activating or enhancing expression in a plant cell, preferably a monocotyledonous or dicotyledonous plant cell and in particular a dicotyledonous plant cell, tissue, or organ. Accordingly, it is within the scope of the invention to include any promoter sequences that also function in non-plant cells, such as yeast cells, animal cells and the like.

The terms “plant-operable” and “operable in a plant” when used herein, in respect of a promoter sequence, shall be taken to be equivalent to a plant-expressible promoter sequence.

In the present context, a “regulatable promoter sequence” is a promoter that is capable of conferring expression on a structural gene in a particular cell, tissue, or organ or group of cells, tissues or organs of a plant, optionally under specific conditions, however does generally not confer expression throughout the plant under all conditions. Accordingly, a regulatable promoter sequence may be a promoter sequence that confers expression on a gene to which it is operably connected in a particular location within the plant or alternatively, throughout the plant under a specific set of conditions, such as following induction of gene expression by a chemical compound or other elicitor.

Preferably, the regulatable promoter used in the performance of the present invention confers expression in a specific location within the plant, either constitutively or following induction, however not in the whole plant under any circumstances. Included within the scope of such promoters are cell-specific promoter sequences, tissue-specific promoter sequences, inducible promoter sequences, organ-specific promoter sequences, cell cycle specific gene promoter sequences, and constitutive promoter sequences that have been modified to confer expression in a particular part of the plant at any one time, such as by integration of said constitutive promoter within an excisable genetic element.

The term “cell-specific” shall be taken to indicate that expression is predominantly in a particular plant cell or plant cell-type, albeit not necessarily exclusively in that plant cell or plant cell-type.

Similarly, the term “tissue-specific” shall be taken to indicate that expression is predominantly in a particular plant tissue or plant tissue-type, albeit not necessarily exclusively in that plant tissue or plant tissue-type.

Similarly, the term “organ-specific” shall be taken to indicate that expression is predominantly in a particular plant organ albeit not necessarily exclusively in that plant organ.

Those skilled in the art will be aware that an “inducible promoter” is a promoter the transcriptional activity of which is increased or induced in response to a developmental, chemical, environmental, or physical stimulus.

Similarly, the term “cell cycle specific” shall be taken to indicate that expression is predominantly cyclic and occurring in one or more, not necessarily consecutive phases of the cell cycle albeit not necessarily exclusively in cycling cells.

As will be apparent from the preceding description, the present invention does not require the exclusive expression of the cell cycle control protein in a cell, tissue or organ of a plant, in order to induce one or more non-pleiotropic cytokinin-mediated and/or gibberellin-mediated effects therein, subject to the proviso that expression is at least predominantly localised in a particular cell, tissue or organ of the plant. Preferably, the promoter selected for regulating expression of the cell cycle control protein in the plant cell, tissue or organ, will confer expression in a range of cell-types or tissue-types or organs, sufficient to produce the desired phenotype, whilst avoiding undesirable phenotypes produced in other cell-types or tissue-types or organs.

More preferably, the promoter selected for regulating expression of the cell cycle control protein in the plant cell, tissue or organ, will confer expression in a limited number of cells or cell-types or tissues or tissue-types or organs of the plant.

Even more preferably, the promoter selected for regulating expression of the cell cycle control protein in the plant cell, tissue or organ, will confer expression in a single cell-type or tissue-type or organ of the plant.

In this regard, those skilled in the art are aware that constitutive promoters confer expression in a wide range of different tissues of plants. Such constitutive promoters are only used to perform the present invention to the extent that they are either not able to confer expression in all tissues of the plant at all times (i.e. independent of developmental and/or environmental stimuli), or alternatively, have been modified to make them “regulatable promoters” within the meaning of the presently described invention. For example, use of a CaMV 35S promoter sequence other than a CaMV minimal promoter sequence that is subject to such modification is not encompassed by the present invention. In this respect, “minimal promoter” means a truncated promoter having only sufficient nucleotide sequence of the base promoter to produce a functional chimeric promoter when modified by the addition or insertion of one or more heterologous promoter sequences, such as, for example, a dexamethasone-responsive GRE.

To make a constitutive promoter sequence tissue-specific, cell-specific, cell-cycle specific or organ-specific, one or more regulatory elements from a regulated promoter may be added to the constitutive promoter sequence. Alternatively, the otherwise constitutive expression activity of the promoter sequence may be regulated by integrating the promoter sequence and cell cycle control gene in one or more excisable genetic elements.

As used herein, the term “an excisable genetic element” shall be taken to refer to any nucleic acid which comprises a nucleotide sequence which is capable of integrating into the nuclear, mitochondrial, or plastid genome of a plant, and subsequently being autonomously mobilised, or induced to mobilise, such that it is excised from the original integration site in said genome. By “autonomously mobilised” is meant that the genetic element is excised from the host genome randomly, or without the application of an external stimulus to excise. In performing the present invention, the genetic element is preferably induced to mobilise, such as, for example, by the expression of a recombinase protein in the cell which contacts the integration site of the genetic element and facilitates a recombination event therein, excising the genetic element completely, or alternatively, leaving a “footprint”, generally of about 20 nucleotides in length or greater, at the original integration site.

Preferably, the excisable genetic element comprises a transposable genetic element, such as, for example, Ac, Ds, Spm, or En, or alternatively, on or more loci for interaction with a site-specific recombinase protein, such as, for example, one or more lox or frt nucleotide sequences.

Known site-specific recombination systems, for example the cre/lox system and the flp/frt system which comprise a loci for DNA recombination flanking a selected gene, specifically lox or frt genetic sequences, combination with a recombinase, cre or flp, which specifically contacts said loci, producing site-specific recombination and deletion of the selected gene. In particular, European Patent No. 0228009 (E. I. Du Pont de Nemours and Company) published Apr. 29, 1987 discloses a method for producing site-specific recombination of DNA in yeast utilising the crellox system, wherein yeast is transformed with a first DNA sequence comprising a regulatory nucleotide sequence and a cre gene and a second DNA sequence comprising a pre-selected DNA segment flanked by two lox sites such that, upon activation of the regulatory nucleotide sequence, expression of the cre gene is effected thereby producing site-specific recombination of DNA and deletion of the pre-selected DNA segment. U.S. Pat. No. 4,959,317 (E. I. Du Pont de Nemours and Company) filed Apr. 29, 1987 and International Patent Application No. PCT/US90/07295 (E. I. Du Pont de Nemours and Company) filed Dec. 19, 1990 also disclose the use of the cre/lox system in eukaryotic cells.

A requirement for the operation of site-specific recombination systems is that the loci for DNA recombination and the recombinase enzyme contact each other in vivo, which means that they must both be present in the same cell. The prior art means for excising unwanted transgenes from genetically-transformed cells all involve either multiple transformation events or sexual crossing to produce a single cell comprising both the loci for DNA recombination and the site-specific recombinase.

A “site-specific recombinase” is understood by those skilled in the relevant art to mean an enzyme or polypeptide molecule which is capable of binding to a specific nucleotide sequence, in a nucleic acid molecule preferably a DNA sequence, hereinafter referred to as a “recombination locus” and induce a cross-over event in the nucleic acid molecule in the vicinity of said recombination locus. Preferably, a site-specific recombinase will induce excision of intervening DNA located between two such recombination loci.

The terms “recombination locus” and “recombination loci” shall be taken to refer to any sequence of nucleotides which is recognized and/or bound by a site-specific recombinase as hereinbefore defined.

A number of different site specific recombinase systems can be used, including but not limited to the Cre/lox system of bacteriophage P1, the FLP/FRT system of yeast, the Gin recombinase of phase Mu, the Pin recombinase of E. coli, the PinB, PinD and PinF from Shigella, and the R/RS system of the psR1 plasmid. Some of these systems have already been used with high efficiency in plants, such as tobacco, and A. thaliana.

Preferred site-specific recombinase systems contemplated for use in the gene constructs of the invention, and in conjunction with the inventive method, are the bacteriophage P1 Cre/lox system, and the yeast FLP/FRT system. The site specific recombination loci for each of these two systems are relatively short, only 34 bp for the lox loci, and 47 bp for the frt loci.

In a most particularly preferred embodiment, however, the recombination loci are lox sites, such as lox P, lox B, Lox L or lox R or functionally-equivalent homologues, analogues or derivatives thereof. Lox sites may be isolated from bacteriophage or bacteria by methods known in the art (Hoess et al., 1982). It will also be known to those skilled in the relevant art that lox sites may be produced by synthetic means, optionally comprising one or more nucleotide substitutions, deletions or additions thereto.

The relative orientation of two recombination loci in a nucleic acid molecule or gene construct may influence whether the intervening genetic sequences are deleted or excised or, alternatively, inverted when a site-specific recombinase acts thereupon. In a particularly preferred embodiment of the present invention, the recombination loci are oriented in a configuration relative to each other such as to promote the deletion or excision of intervening genetic sequences by the action of a site-specific recombinase upon, or in the vicinity of said recombination loci.

The present invention clearly encompasses the use of gene constructs which facilitate the expression of a site-specific recombinase protein which is capable of specifically contacting the excisable genetic element, in conjunction with the gene constructs containing the cell cycle control protein-encoding gene. A single gene construct may be used to express both the site-specific recombinase protein and the cell cycle control protein, or alternatively, these may be introduced to plant cells on separate gene constructs.

For example, the recombinase gene could already be present in the plant genome prior to transformation with the gene construct of the invention, or alternatively, it may be introduced to the cell subsequent to transformation with the gene construct of the invention, such as, for example, by a separate transformation event, or by standard plant breeding involving hybridisation or cross-pollination. In one embodiment of the current invention, the recombinase gene is supplied to the transgenic plants containing a vector backbone sequence flanked by recombination sites by sexual crossing with a plant containing the recombinase gene in it's genome. Said recombinase can be operably linked to either a constitutive or an inducible promoter. The recombinase gene can alternatively be under the control of single subunit bacteriophage RNA polymerase specific promoters, such as a T7 or a T3 specific promoter, provided that the host cells also comprise the corresponding RNA polymerase in an active form. Yet another alternative method for expression of the recombinase consists of operably linking the recombinase open reading frame with an upstream activating sequence fired by a transactivating transcription factor such as GAL4 or derivatives (U.S. Pat. No. 5,801,027, WO97/30164, WO98/59062) or the Lac repressor (EP0823480), provided that the host cell is supplied in an appropriate way with the transcription factor.

Alternatively, a substantially purified recombinase protein could be introduced directly into the eukaryotic cell, eg., by micro-injection or particle bombardment. Typically, the site-specific recombinase coding region will be operably linked to regulatory sequences enabling expression of the site-specific recombinase in the eukaryotic cell. In a preferred embodiment of the present invention, the site-specific recombinase sequences is operably linked to an inducible promoter.

Dual-specific recombinase systems can also be employed, which may employ a recombinase enzyme in conjunction with direct or indirect repeats of two different site-specific recombination loci corresponding to the dual-specific recombinase, such as that described in International Patent Publication No. WO99/25840.

As will be known to those skilled in the art, for recombination mediated by a transposon to occur, a pair of DNA sequences comprising inverted repeat transposon border sequences, flanking the excisable genetic element sequence, and a specific transposase enzyme, are required. The transposase catalyzes a recombination reaction only between two transposon border sequences.

A number of different plant-operable transposon/transposase systems can be used including but not limited to the Ac/Ds system, the Spm system and the Mu system. All of these systems are operable in Zea mays, and at least the Ac/Ds and the Spm system function in other plants.

Preferred transposon sequences for use in the gene constructs of the invention are the Ds-type and the Spm-type transposons, which are delineated by border sequences of only 11 bp and 13 bp in length, respectively.

As with the use of site-specific recombinase systems, the present invention clearly encompasses the use of gene constructs which facilitate the expression of a transposase enzyme which is capable of specifically contacting the transposon border sequence, in conjunction with the gene constructs containing thecell cycle control protein-encoding gene. A single gene construct may be used to express both the transposase and the cell cycle control protein, or alternatively, these may be introduced to plant cells on separate gene constructs.

For example, the transposase-encoding gene could already be present in the plant genome prior to transformation with the gene construct of the invention, or alternatively, it may be introduced to the cell subsequent to transformation with the gene construct of the invention, such as, for example, by a separate transformation event, or by standard plant breeding involving hybridisation or cross-pollination. Alternatively, a substantially purified transposase protein could be introduced directly into the eukaryotic cell, eg., by micro-injection or particle bombardment. Typically, the transposase coding region will be operably linked to regulatory sequences enabling expression of the transposase in the eukaryotic cell. In a preferred embodiment of the present invention, the transposase-encoding sequence is operably linked to an inducible promoter.

In the present context, transposon border sequences are organized as inverted repeats flanking the excisable genetic element. As transposons often re-integrate at another locus of the host s genome, segregation of the progeny of the hosts in which the transposase was allowed to act might be necessary to separate transformed hosts containing only the gene(s) of interest and transformed hosts containing only the cell cycle control protein-encoding gene.

Likewise, the site-specific recombinase gene or transposase gene present in the host's genome can be removed by segregation of the progeny of the hosts to separate transformed hosts containing only the gene(s) of interest and transformed hosts containing only the site-specific recombinase gene or transposase gene. Alternatively, said site-specific recombinase gene or transposase gene are included in the same or in a different excisable genetic element as thecell cycle control protein-encoding gene.

Those skilled in the art will readily be capable of selecting appropriate promoter sequences for use in regulating appropriate expression of the cell cycle control protein from publicly-available or readily-available sources, without undue experimentation.

Placing a nucleic acid molecule under the regulatory control of a promoter sequence, or in operable connection with a promoter sequence, means positioning said nucleic acid molecule such that expression is controlled by the promoter sequence.

A promoter is usually, but not necessarily, positioned upstream, or at the 5′-end, and within 2 kb of the start site of transcription, of the nucleic acid molecule which it regulates.

In the construction of heterologous promoter/structural gene combinations it is generally preferred to position the promoter at a distance from the gene transcription start site that is approximately the same as the distance between that promoter and the gene it controls in its natural setting (i.e., the gene from which the promoter is derived). As is known in the art, some variation in this distance can be accommodated without loss of promoter function. Similarly, the preferred positioning of a regulatory sequence element with respect to a heterologous gene to be placed under its control is defined by the positioning of the element in its natural setting (i.e., the gene from which it is derived). Again, as is known in the art, some variation in this distance can also occur.

Examples of promoters suitable for use in gene constructs of the present invention include those listed in Table 1, amongst others. The promoters listed in Table 1 are provided for the purposes of exemplification only and the present invention is not to be limited by the list provided therein. As explained herein, the constitutive promoters referred to herein are preferably subjected to modification to render them regulatable. Those skilled in the art will readily be in a position to provide additional promoters that are useful in performing the present invention.

In an alternative embodiment, the promoter is a tissue-specific inducible promoter sequence, such as but not limited to a light-inducible rbcs-1A or rbcs-3A promoter, anoxia-inducible maize Adh1 gene promoter (Howard et al., 1987; Walker et al., 1987), hypoxia-inducible maize Adh1 gene promoter (Howard et al., 1987; Walker et al., 1987), and the temperature-inducible heat shock promoter. Such environmentally-inducible promoters are reviewed in detail by Kuhlemeier et al. 1987).

In an alternative embodiment, the promoter is a chemically-inducible promoter, such as the 3- - indoylacrylic acid-inducible Tip promoter; IPTG-inducible lac promoter; phosphate-inducible promoter; L-arabinose-inducible araB promoter; heavy metal-inducible metallothionine gene promoter; dexamethasone-inducible promoter; glucocorticoid-inducible promoter; ethanol-inducible promoter (Zeneca); the N,N-diallyl-2,2-dichloroacetamide-inducible glutathione-S-transferase gene promoter (Wiegand et al., 1986); or any one or more of the chemically-inducible promoters described by Gatz et al. (1996), amongst others.

In an alternative embodiment, the promoter is a wound-inducible or pathogen-inducible promoter, such as the phenylalanine ammonia lyase (PAL) gene promoter (Ebel et al., 1984), chalcone synthase gene promoter (Ebel et al., 1984) or the potato wound-inducible promoter (Cleveland et a., 1987), amongst others.

In a further alternative embodiment, the promoter is a hormone-inducible promoter, such as the abscisic acid-inducible wheat 7S globulin gene promoter and the wheat Em gene promoter (Marcotte et al.,1988);an auxin-responsive gene promoter, such as, for example, the SAUR gene promoter, the parAs and parAt gene promoters(van der Zaal et al., 1991; Gil et al., 1994; Niwa et al., 1994); or a gibberellin-inducible promoter such as the Amy32b gene promoter (Lanahan et al. 1992), amongst others.

In a further alternative embodiment, the promoter is derived from a constitutive plant-expressible promoter sequence such as the CaMV 35S promoter sequence, CaMV 19S promoter sequence, the octopine synthase (OCS) promoter sequence, or nopaline synthase (NOS) promoter sequence (Ebert et al. 1987), amongst others.

In the case of constitutive promoters or promoters that induce expression throughout the entire plant, it is preferred that such sequences are modified by the addition of nucleotide sequences derived from one or more of the tissue-specific promoters listed in Table 1, or alternatively, nucleotide sequences derived from one or more of the above-mentioned tissue-specific inducible promoters, to confer tissue-specificity thereon. For example, the CaMV 35S promoter may be modified by the addition of maize Adh1 promoter sequence, to confer anaerobically-regulated root-specific expression thereon, as described previously (Ellis et al., 1987). Such modifications can be achieved by routine experimentation by those skilled in the art.

In yet another alternative embodiment, the promoter is a cell cycle specific gene promoter, such as, for example, the Cdc2a promoter sequence (Chung and Parish 1995) or the PCNA promoter sequence (Kosugi et al, 1991, Kosugi and Ohashi 1997).

Preferred embodiments of the invention relate to the effect(s) of cytokinins on plant morphology and architecture. The present invention clearly contemplates the broad application of the inventive method to the modification of a range of cellular processes, including but not limited to the modification of initiation, promotion, stimulation or enhancement of cell division and/or seed development and/or tuber formation and/or shoot initiation and/or bushiness and/or dwarfism and/or pigment synthesis, and/or the modification of source/sink relationships, and/or the inhibition of root growth and/or the inhibition of apical dominance and/or the delay of senescence. In this regard, the identification of substrates of Cdc25 phosphatase other than Cdc2 will also reveal the mechanism by which Cdc25 is linked to many cellular processes other than cell division.

In a particularly preferred embodiment of the present invention, there is provided a method of increasing the strength and/or thickness and/or stability and/or wind-resistance of a plant comprising expressing the yeast Cdc25 protein or a homologue, analogue or derivative thereof operably under the control of a stem-expressible promoter sequence.

Preferably, the stem-expressible promoter sequence is derived from the rbcs-1A gene, the rbcs-3A gene, the AtPRP4 gene, the T. bacilliform virus gene, or the sucrose-binding protein gene set forth in Table 1, or a stem-specific or stem-expressible homologue, analogue or derivative thereof.

In another preferred embodiment of the present invention, there is provided a method of increasing tuber formation and/or development in a tuberous crop plant comprising expressing the yeast Cdc25 protein or a homologue, analogue or derivative thereof operably under the control of a tuber-specific promoter sequence.

Preferably, the tuberous crop plant is potato and the tuber-specific promoter is the potato patatin gene promoter. Additional species and promoters are not excluded.

In another preferred embodiment of the present invention, there is provided a method of modifying the lignin content of a woody crop plant comprising expressing the yeast Cdc25 protein or a homologue, analogue or derivative thereof operably under the control of a cambium-specific or vascular-tissue-specific promoter sequence.

Preferably, the promoter is a cinnamoyl alcohol dehydrogenase (CAD) gene promoter, laccase gene promoter, cellulose synthase gene promoter and xyloglucan endotransglucosylase (XET) gene promoter sequences, amongst others. The T. bacilliform virus gene promoter and the sucrose-binding protein gene promoter are also useful for this application of the invention.

Preferred target plant species according to this embodiment are woody plants of economic/ agronomic value, in particular hardwood crop plants such as, but not limited to Eucalyptus spp., Populus spp., Quercus spp., Acer spp., Juglans spp., Fagus spp., Acacia spp., or teak, amongst others. More preferably, this embodiment of the invention is applicable to modifying the lignin content of Eucalyptus spp., in particular E. globulus and E. robusta; or Quercus spp., in particular Q. dentata, Q. ilex, Q. incana, and Q. robur; Acacia spp., in particular A. brevispica, A. bussei, A. drepanolobium, A. nilotica, A. pravissima, and A. seyal, Acer spp., in particular A. pseudoplatanus and A. saccharum. Additional species are not excluded.

Without being bound by any theory or mode of action, the ectopic expression of Cdc25 under control of a promoter that is operable in vascular tissue and preferably, in cambial cells, will produce thick-stemmed plants and a higher ratio of vascular tissue-to-pith cells within the stem, thereby resulting in more lignin production. Within the vascular tissue, cambial cells contain the highest levels of auxins and are therefore the preferential tissue for Cdc25 overproduction.

In yet another preferred embodiment of the present invention, there is provided a method of increasing seed set and/or seed production and/or seed size and/or grain yield in a plant comprising expressing the yeast Cdc25 protein or a homologue, analogue or derivative thereof operably under the control of a seed-specific promoter sequence.

Preferably, the seed-specific promoter is operable in the seeds of monocotyledonous plants, for example the barley Amy32b gene promoter, Cathepsin -like gene promoter, wheat ADP-glucose pyrophosphorylase gene promoter, maize zein gene promoter, or rice glutelin gene promoter. In an alternative embodiment, the seed-specific promoter is operable in the seeds of dicotyledonous plant species, for example the legumin gene promoter, napA gene promoter, Brazil Nut albumin gene promoter, pea vicilin gene promoter and sunflower oleosin gene promoter, amongst others.

Those skilled in the art will be aware that grain yield in crop plants is largely a function of the amount of starch produced in the endosperm of the seed. The amount of protein produced in the endosperm is also a contributing factor to grain yield. In contrast, the embryo and aleurone layers contribute little in terms of the total weight of the mature grain. By virtue of being linked to cell expansion and metabolic actvity, endoreplication and endoreduplication are generally considered as an important factor for increasing yield (Traas et al 1998). As grain endosperm development initially includes extensive endoreplication (Olsen et al 1999), enhancing, promoting or stimulating this process is likely to result in increased grain yield. Enhancing, promoting or stimulating cell division during seed development is an alternative way to increase grain yield. As shown in the present invention Cdc25 is involved both in the regulation of endoreplucation and in the stimulation of cell division.

The promoters listed in Table 1 are provided for the purposes of exemplification only and the present invention is not to be limited by the list provided therein. Those skilled in the art will readily be in a position to provide additional promoters that are useful in performing the present invention. The promoters listed may also be modified to provide specificity of expression as required.

TABLE 1
EXEMPLARY PLANT-EXPRESSIBLE PROMOTERS FOR USE IN THE PERFORMANCE
OF THE PRESENT INVENTION

| | | |
|---|---|---|
| GENE SOURCE | EXPRESSION PATTERN | REFERENCE |

I: CELL-SPECIFIC, TISSUE-SPECIFIC, AND ORGAN-SPECIFIC PROMOTERS

| α-amylase (Amy32b) | Aleurone | Lanahan et al., 1992; Skriver et al., 1991 |
|---|---|---|
| cathepsin β-like gene | Aleurone | Cejudo et al., 1992. |
| Agrobacterium rhizogenes rolB | Cambium | Nilsson et al., 1997 |
| PRP genes | cell wall | http://salus.medium.edu/mmg/tierney/html |
| AtPRP4 | Flowers | http://salus.medium.edu/mmg/tierney/html |
| chalene synthase (chsA) | Flowers | Van der Meer et al., 1990. |
| LAT52 | Anther | Twell et al, 1989 |
| apetala-3 | Flowers | |
| Chitinase | fruit (berries, grapes, etc) | Thomas et al. CSIRO Plant Industry, Urrbrae, South |
| | | Australia, Australia; http://winetitles.com.au/gwrdc/csh95- |
| | | 1.html |
| rbcs-3A | green tissue (eg leaf) | Lam et al., 1990; Tucker et al., 1992. |
| leaf-specific genes | Leaf | Baszczynski et al., 1988. |
| AtPRP4 | Leaf | http://salus.medium.edu/mmg/tierney/html |
| Pinus cab-6 | Leaf | Yamamoto et al., 1994. |
| SAM22 | senescent leaf | Crowell et al., 1992. |
| R. japonicum nif gene | Nodule | U.S. Pat. No. 4,803,165 |
| B. japonicum nifH gene | Nodule | U.S. Pat. No. 5,008,194 |
| GmENOD40 | Nodule | Yang et al, 1993. |
| PEP carboxylase (PEPC) | Nodule | Pathirana et al., 1992. |
| leghaemoglobin (Lb) | Nodule | Gordon et al., 1993. |
| Tungro bacilliform virus gene | Phloem | Bhattacharyya-Pakrasi et al, 1992. |
| sucrose-binding protein gene | plasma membrane | Grimes et al., 1992. |
| pollen-specific genes | pollen; microspore | Albani et al., 1990; Albani et al., 1991 |
| Zm13 | Pollen | Guerrero et al, 1993 |
| apg gene | Microspore | Twell et al, 1993 |
| maize pollen-specific gene | Pollen | Hamilton et al., 1992. |
| sunflower pollen-expressed gene | Pollen | Baltz et al., 1992. |
| B. napus pollen-specific gene | pollen; anther; tapetum | Arnoldo et al., 1992. |
| root-expressible genes | Roots | Tingey et al., 1987. |
| tobacco auxin-inducible gene | root tip | Van der Zaal et al., 1991. |
| β-tubulin | Root | Oppenheimer et al., 1988. |
| tobacco root-specific genes | Root | Conkling et al., 1990. |
| B. napus G1-3b gene | Root | U.S. Pat. No. 5,401,836 |
| SbPRP1 | Root | Suzuki et al., 1993, |
| AtPRP1; AtPRP3 | roots; root hairs | http://salus.medium.edu/mmg/tierney/html |
| RD2 gene | root cortex | http://www2.cnsu.edu/ncsu/research |
| TobRB7 gene | root vasculature | http://www2.cnsu.edu/ncsu/research |
| AtPRP4 | leaves; flowers; lateral root | http://salus.medium.edu/mmg/tierney/html |
| | primordia | |
| seed-specific genes | Seed | Simon et al., 1985; Scofield et al., 1987; Baszczynski et al., |
| | | 1990. |
| Brazil Nut albumin | Seed | Pearson et al., 1992. |
| Legumin | Seed | Ellis et al., 1988. |
| glutelin (rice) | Seed | Takaiwa et al., 1986; Takaiwa et al., 1987. |
| Zein | Seed | Matzke et al, 1990 |
| NapA | Seed | Stalberg et al, 1996. |
| wheat LMW and HMW glutenin-1 | Endosperm | Mol Gen Genet 216:81-90, 1989; NAR 17:461-2, 1989 |
| wheat SPA | Seed | Albani et al, 1997 |
| wheat α, β, γ-gliadins | Endosperm | EMBO 3:1409-15, 1984 |
| barley ltr1 promoter | Endosperm | |
| barley B1, C, D, hordein | Endosperm | Theor Appl Gen 98:1253-62, 1999; Plant J 4:343-55, 1993; |
| | | Mol Gen Genet 250:750-60, 1996 |
| barley DOF | Endosperm | Mena et al, 1998 |
| blz2 | Endosperm | EP99106056.7 |
| synthetic promoter | Endosperm | Vicente-Carbajosa et al., 1998. |
| rice prolamin NRP33 | Endosperm | Wu et al, 1998 |
| rice α-globulin Glb-1 | Endosperm | Wu et al, 1998 |
| rice OSH1 | Embryo | Sato et al, 1996 |
| rice α-globulin REB/OHP-1 | Endosperm | Nakase et al., 1997 |
| rice ADP-glucose PP | Endosperm | Trans Res 6:157-68, 1997 |
| maize ESR gene family | Endosperm | Plant J 12:235-46, 1997 |
| sorgum γ-kafirin | Endosperm | PMB 32:1029-35, 1996 |
| KNOX | Embryo | Postma-Haarsma et al, 1999 |
| rice oleosin | embryo and aleurone | Wu et al, 1998 |
| sunflower oleosin | seed (embryo and dry seed) | Cummins et al., 1992 |
| LEAFY | shoot meristem | Weigel et al., 1992. |
| Arabidopsis thaliana knat1 | shoot meristem | Accession number AJ131822 |
| Malus domestica kn1 | shoot meristem | Accession number Z71981 |
| CLAVATA1 | shoot meristem | Accession number AF049870 |
| stigma-specific genes | Stigma | Nasrallah et al., 1988; Trick et al., 1990 |
| class l patatin gene | Tuber | Liu et al., 1991. |
| PCNA rice | Meristem | Kosugi et al, 1991; Kosugi et al., 1997. |
| Pea TubA1 tubulin | Dividing cells | Stotz et al., 1999 |
| Arabidopsis Cdc2a | cycling cells | Chung and Parish, 1995 |
| Arabidopsis Rop1A | Anthers; mature pollen; pollen | Li et al, 1998 |
| | tube | |
| Arabidopsis AtDMC1 | Melosis-associated | Klimyuk and James, 1997 |
| Pea PS-IAA4/5 and PS-IAA6 | Auxin-inducible | Wong et al., 1996 |
| Pea farnesyltransferase | Meristematic tissues; phloem | Zhou et al., 1997 |
| | near growing tissues; light-and | |
| | sugar-repressed | |
| Tobacco (N. sylvestris) cyclin | Dividing cells/meristematic | Trehin et al., 1997 |
| B1; 1 | tissue | |
| Catharanthus roseus | Dividing cells/meristematic | Ito et al., 1997 |
| Mitotic cyclins CYS (A-type) and CYM | tissue | |
| (B-type) | | |
| Arabidopsis cyc1At (= cyc B1; 1) | Dividing cells/meristematic | Shaul et al., 1996 |
| and cyc3aAt (A-type) | tissue | |
| Arabidopsis tef1 promoter box | Dividing cells/meristematic | Regad et al., 1995 |
| | tissue | |
| Catharanthus roseus cyc07 | Dividing cells/meristematic | Ito et al., 1994 |
| | tissue | |
| GENE SOURCE | EXPRESSION PATTERN | REFERENCE |

II: EXEMPLARY CONSTITUTIVE PROMOTERS

| Actin | Constitutive | McElroy et al, 1990 |
|---|---|---|
| CAMV 35S | Constitutive | Odell et al, 1985 |
| CaMV 19S | Constitutive | Nilsson et al., 1997 |
| GO52 | Constitutive | de Pater et al, 1992 |
| Ubiquitin | Constitutive | Christensen et al, 1992 |
| rice cyclophilin | Constitutive | Buchholz et al, 1994 |
| maize H3 histone | Constitutive | Lepetit et al, 1992 |
| actin 2 | Constitutive | An et al, 1996 |
| NAME | STRESS | REFERENCE |

III: EXEMPLARY STRESS-INDUCIBLE PROMOTERS

| P5CS (delta(1)-pyrroline-5-carboxylate | salt, water | Zhang et al, 1997 |
|---|---|---|
| synthase) | | |
| cor15a | cold | Hajela et al., 1990 |
| cor15b | cold | Wilhelm and Thomashow, 1993 |
| cor15a (−305 to +78 nt) | cold, drought | Baker et al., 1994 |
| rd29 | salt, drought, cold | Kasuga et al., 1999 |
| heat shock proteins, including | heat | Barros et al., 1992. Marrs et al., 1993. Schoffl et al., 1989. |
| artificial promoters containing the | | |
| heat shock element (HSE) | | |
| smHSP (small heat shock proteins) | heat | Waters et al, 1996 |
| wcs120 | cold | Quellet et al., 1998 |
| ci7 | cold | Kirch et al., 1997 |
| Adh | cold, drought, hypoxia | Dolferus et al., 1994 |
| pwsi18 | water: salt and drought | Joshee et al., 1998 |
| ci21A | cold | Schneider et al., 1997 |
| Trg-31 | drought | Chaudhary et al., 1996 |
| Osmotin | osmotic | Raghothama et al., 1993 |
| LapA | wounding, enviromental | W099/03977 University of California/INRA |
| NAME | PATHOGEN | REFERENCE |

IV: EXEMPLARY PATHOGEN-INDUCIBLE PROMOTERS

| | | |
|---|---|---|
| RB7 | Root-knot nematodes | U55760386-North Carolina State University; Opperman et |
| | (Meloidogyne spp.) | al, 1994 |
| PR-1, 2, 3, 4, 5, 8, 11 | fungal, viral, bacterial | Ward et al., 1991; Reiss and Bryngelsson, 1996; Lebel et |
| | | al., 1998; Melchers et al., 1994; Lawton et al., 1992 |
| HMG2 | Nematodes | W09503690-Virginia Tech Intellectual Properties Inc. |
| Abi3 | Cyst nematodes (Heterodera | unpublished |
| | spp.) | |
| ARM1 | Nematodes | Barthels et al., 1997; WO 98/31822 Plant Genetic Systems |
| Att0728 | Nematodes | Barthels et al., 1997; PCT/EP98/07761 |
| Att1712 | Nematodes | Barthels et al., 1997; PCT/EP98/07761 |
| Gstl | Different types of pathogens | Strittmatter et al., 1996 |
| LEMMl | Nematodes | WO 92/21757-Plant Genetic Systems |
| CLE | Geminivirus | PCT/EP99103445-CINESTAV |
| PDF1.2 | Fungal including Alternaria | Manners et al., 1998 |
| | brassicicola and Botiytis | |
| | cinerea | |
| Thi2.1 | Fungal-Fusarium oxysporum f | Vignutelli et al., 1998 |
| | sp. Matthiolae | |
| DB#226 | Nematodes | Bird and Wilson, 1994; WO 95.322888 |
| DB#280 | Nematodes | Bird and Wilson, 1994; WO 95.322888 |
| Cat2 | Nematodes | Niebel et al., 1995 |
| tub | Nematodes | Aristizabal et al (1996), 8^{th} International Congress on Plant- |
| | | Microbe Interaction, Knoxville U.S. B-29 |
| SHSP | Nematodes | Fenoll et al., 1997 |
| Tsw12 | Nematodes | Fenoll et al., 1997 |
| Hs1 (prol) | Nematodes | WO 98/122335-Jung |
| NsLTP | viral, fungal, bacterial | Molina et al., 1993 |
| RIP | viral, fungal | Tumer et al., 1997 |

Accordingly, in a preferred embodiment, the Cdc25 gene is placed operably in connection with a promoter that is operable in the endosperm of the seed, in which case the combination of the cell cycle-control protein and endosperm-expressible promoter provides the additional advantage of increasing the grain size and grain yield of the plant.

Endosperm-specific promoters that can be used to drive Cdc25 expression have been identified. The components of the promoters responsible for specific expression have been identified (Grosset et al (1997) and are interchangeable between agriculturally important cereals (Olsen et al 1992; Russell and Fromm, 1997). Several promoters can be used, including the barley biz2 gene promoter, the rice prolamin NRP33 promoter, the rice REB promoter, the zein (ZmZ27) gene promoter, the rice glutelin 1 gene (osGT1) promoter, the rice small subunit ADP-glucose pyrophosphorylase (osAGP) promoter, the maize granule-bound starch synthase (Waxy) gene (zmGBS) promoter surveyed by Russell and Fromm (1997), the Brazil Nut albumin gene promoter, and the pea vicilin gene promoter, amongst others. Promoters derived from those genes that are expressed in the endosperm during nuclear proliferation are also useful for driving Cdc25 expression. Promoters derived from those genes that are expressed in the endosperm at the stage when nuclear proliferation is ending could be ideal for extending this period.

A three way correlation exists between cytokinin level in the endosperm, the number of endosperm cells formed during seed development and grain size, in which cytokinin activates Cdc25 enzyme which in turn activates Cdc2 kinase to drive nuclear division. Accordingly, ectopic expression of the Cdc25 gene in the endosperm enhances Cdc2 activation and nuclear proliferation, resulting in increased grain size, without incurring the non-specific side effects that application of cytokinin or expression of the ipt gene would produce in the plant.

A further advantage of the present inventive approach is that the activity of cytokinin metabolising enzymes is circumvented by the direct raising of Cdc25 activity in the endosperm, by the ectopic expression of Cdc25 therein. In cases where exogenous cytokinin is used to increase grain size and/or endosperm size, the elevated cytokinin levels and nuclear division in the grain are curtailed by an increase in the activities of cytokinin degrading enzymes, including cytokinin oxidase (Chaffield and Armstrong 1987; reviewed by Morris et al 1993).

In another preferred embodiment of the present invention, there is provided a method of inhibiting or reducing apical dominance or increasing the bushiness of a plant, comprising expressing the yeast Cdc25 protein or a homologue, analogue or derivative thereof operably under the control of a meristem-specific promoter sequence or a stem-specific promoter sequence.

Without being bound by any theory or mode of action, increased cell division in the dormant lateral meristem of plants as a consequence of increased Cdc25 activity therein results in a higher degree of branch formation in the plant, thereby alleviating auxin-induced apical dominance in the plant.

In another preferred embodiment of the present invention, there is provided a method of increasing lateral root production in a plant comprising expressing the yeast Cdc25 protein or a homologue, analogue or derivative thereof operably under the control of a root-specific promoter sequence.

Preferred promoter sequences according to this embodiment of the present invention include any one of the root-expressible or root-specific promoters listed in Table 1 and in particular, the tobacco auxin-inducible gene promoter described by Van der Zaal et al (1991) that confers expression in the root tip of plants, in particular dicotyledonous plants.

In yet another preferred embodiment of the present invention, there is provided a method of increasing the nitrogen-fixing capability of a plant comprising expressing the yeast Cdc25 protein or a homologue, analogue or derivative thereof operably under the control of a nodule-specific promoter sequence.

Preferred nodule-specific promoter sequences according to this embodiment of the present invention are listed in Table 1. Additional promoters that are suited for this purpose include the hemoglobin gene promoters derived from Frankia spp., A. thaliana or other plants.

In still another preferred embodiment of the present invention, there is provided a method of preventing and/or delaying and/or otherwise reducing leaf chlorosis and/or leaf necrosis in a plant comprising expressing the yeast Cdc25 protein or a homologue, analogue or derivative thereof operably under the control of a leaf-specific promoter sequence.

Preferred promoters for use according to this embodiment of the present invention include the SAM22 promoter, rbcs-1A and rbcs-3A gene promoters listed in Table 1. The SAM22 gene promoter is particularly preferred in light of the developmental regulation of the SAM22 gene and its induction in senescent leaves.

In a further preferred embodiment of the present invention, the yeast Cdc25 protein or a homologue analogue or derivative thereof is expressed in one of the specialised minority of plant tissues in which the activation of cell cycle progression that is generally contributed by cytokinin is in part performed by other hormones. An example of such a tissue is the youngest stem internode of cereal plants in which gibberellic acid stimulates cell division.

Accordingly, the present invention preferably provides a method of stimulating cell division in the intercalary meristem of the youngest stem internode to produce greater elongation of the stem and/or to generate a more extensive photosynthetic canopy of a plant comprising expressing the yeast Cdc25 protein or a homologue, analogue or derivative thereof operably under the control of a meristem specific promoter sequence.

Without being bound by any theory or mode of action, increase in cell division in the intercalary meristem of the youngest stem internode as a consequence of increased Cdc25 activity therein results in greater vigour of the plant due to stem elongation and the production of a more extensive canopy. It is proposed that this leads to an increase in the plant's capacity to support grain production. The stimulatory effect of gibberellic acid application is thus obtained without side effects on flowering time and seed germination.

Preferred promoters for use according to this embodiment of the invention include meristem promoters listed in Table 1 and in particular the Proliferating Cell Nuclear Antigen (PCNA) promoter of rice described by Kosugi et al (1991).

In each of the preceding embodiments of the present invention, the cell cycle control protein is expressed under the operable control of a regulatable promoter sequence. As will be known those skilled in the art, this is generally achieved by introducing a gene construct or vector into plant cells by transformation or transfection means. The nucleic acid molecule or a gene construct comprising same may be introduced into a cell using any known method for the transfection or transformation of said cell. Wherein a cell is transformed by the gene construct of the invention, a whole organism may be regenerated from a single transformed cell, using any method known to those skilled in the art.

By “transfect” is meant that the gene construct or vector or an active fragment thereof comprising the Cdc25 gene operably under the control of the regulatable promoter sequence is introduced into said cell without integration into the cell's genome.

By “transform” is meant that the gene construct or vector or an active fragment thereof comprising the Cdc25 gene operably under the control of the regulatable promoter sequence is stably integrated into the genome of the cell.

Accordingly, in a further preferred embodiment, the present invention provides a method of modifying one or more plant morphological and/or biochemical and/or physiological characteristics comprising

(i) introducing to a plant cell, tissue or organ a gene construct or vector comprising a nucleotide sequence that encodes a cell cycle control protein, such as, for example, Cdc25 or a homologue, analogue or derivative thereof, operably in connection with a regulatable promoter sequence selected from the list comprising cell-specific promoter sequences, tissue-specific promoter sequences, inducible promoter sequences, organ-specific promoter sequences and cell cycle gene promoter sequences to produce a transformed or transfected cell; and

(ii) expressing said cell cycle control protein in one or more of said cells, tissues or organs of the plant.

In an alternative embodiment, the inventive method comprises regenerating a whole plant from the transformed cell.

Means for introducing recombinant DNA into plant tissue or cells include, but are not limited to, transformation using CaCl₂ and variations thereof, in particular the method described by Hanahan (1983), direct DNA uptake into protoplasts (Krens et al, 1982; Paszkowski et al, 1984), PEG-mediated uptake to protoplasts (Armstrong et al, 1990) microparticle bombardment, electroporation (Fromm et al., 1985), microinjection of DNA (Crossway et al., 1986), microparticle bombardment of tissue explants or cells (Christou et al, 1988; Sanford, 1988), vacuum-infiltration of tissue with nucleic acid, or in the case of plants, T-DNA-mediated transfer from Agrobacterium to the plant tissue as described essentially by An et al.(1985), Herrera-Estrella et al. (1983a, 1983b, 1985).

For microparticle bombardment of cells, a microparticle is propelled into a cell to produce a transformed cell. Any suitable ballistic cell transformation methodology and apparatus can be used in performing the present invention. Exemplary apparatus and procedures are disclosed by Stomp et al. (U.S. Pat. No. 5,122,466) and Sanford and Wolf (U.S. Pat. No. 4,945,050). When using ballistic transformation procedures, the gene construct may incorporate a plasmid capable of replicating in the cell to be transformed.

Examples of microparticles suitable for use in such systems include 1 to 5 m gold spheres. The DNA construct may be deposited on the microparticle by any suitable technique, such as by precipitation.

A whole plant may be regenerated from the transformed or transfected cell, in accordance with procedures well known in the art. Plant tissue capable of subsequent clonal propagation, whether by organogenesis or embryogenesis, may be transformed with a gene construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem).

The term “organogenesis”, as used herein, means a process by which shoots and roots are developed sequentially from meristematic centres.

The term “embryogenesis”, as used herein, means a process by which shoots and roots develop together in a concerted fashion (not sequentially), whether from somatic cells or gametes.

Preferably, the transformed plants are produced by a method that does not require the application of exogenous cytokinin and/or gibberellin during the tissue culture phase, such as, for example, an in planta transformation method. In a particularly preferred embodiment, plants are transformed by an in planta method using Agrobacterium tumefaciens such as that described by Bechtold et al., (1993) or Clough et al (1998), wherein A. tumefaciens is applied to the outside of the developing flower bud and the binary vector DNA is then introduced to the developing microspore and/or macrospore and/or the developing seed, so as to produce a transformed seed without the exogenous application of cytokinin and/or gibberellin. Those skilled in the art will be aware that the selection of tissue for use in such a procedure may vary, however it is preferable generally to use plant material at the zygote formation stage for in planta transformation procedures.

The regenerated transformed plants may be propagated by a variety of means, such as by clonal propagation or classical breeding techniques. For example, a first generation (or T1) transformed plant may be selfed to give homozygous second generation (or T2) transformant, and the T2 plants further propagated through classical breeding techniques.

The regenerated transformed organisms contemplated herein may take a variety of forms. For example, they may be chimeras of transformed cells and non-transformed cells; clonal transformants (e.g., all cells transformed to contain the expression cassette); grafts of transformed and untransformed tissues (e.g., in plants, a transformed root stock grafted to an untransformed scion ).

A further aspect of the present invention clearly provides the gene constructs and vectors designed to facilitate the introduction and/or expression and/or maintenance of the cell cycle control protein-encoding sequence and regulatable promoter into a plant cell, tissue or organ.

In addition to the cell cycle control protein-encoding sequence and regulatable promoter sequence, the gene construct of the present invention may further comprise one or more terminator sequences.

The term “terminator” refers to a DNA sequence at the end of a transcriptional unit which signals termination of transcription. Terminators are 3′-non-translated DNA sequences containing a polyadenylation signal, which facilitates the addition of polyadenylate sequences to the 3′-end of a primary transcript. Terminators active in cells derived from viruses, yeasts, moulds, bacteria, insects, birds, mammals and plants are known and described in the literature. They may be isolated from bacteria, fungi, viruses, animals and/or plants.

Examples of terminators particularly suitable for use in the gene constructs of the present invention include the Agrobacterium tumefaciens nopaline synthase (NOS) gene terminator, the Agrobacterium tumefaciens octopine synthase (OCS) gene terminator sequence, the Cauliflower mosaic virus (CaMV) 35S gene terminator sequence, the Oryza sativa ADP-glucose pyrophosphorylase terminator sequence (t3′Bt2), the Zea mays zein gene terminator sequence, the rbcs-1A gene terminator, and the rbcs-3A gene terminator sequences, amongst others.

Those skilled in the art will be aware of additional promoter sequences and terminator sequences which may be suitable for use in performing the invention. Such sequences may readily be used without any undue experimentation.

The gene constructs of the invention may further include an origin of replication sequence which is required for maintenance and/or replication in a specific cell type, for example a bacterial cell, when said gene construct is required to be maintained as an episomal genetic element (eg. plasmid or cosmid molecule) in said cell.

Preferred origins of replication include, but are not limited to, the f1-ori and co/E1 origins of replication.

The gene construct may further comprise a selectable marker gene or genes that are functional in a cell into which said gene construct is introduced.

As used herein, the term “selectable marker gene” includes any gene which confers a phenotype on a cell in which it is expressed to facilitate the identification and/or selection of cells which are transfected or transformed with a gene construct of the invention or a derivative thereof.

Suitable selectable marker genes contemplated herein include the ampicillin resistance (Amp′), tetracycline resistance gene (Tc′), bacterial kanamycin resistance gene (Kan′), phosphinothricin resistance gene, neomycin phosphotransferase gene (nptII), hygromycin resistance gene, β-glucuronidase (GUS) gene, chloramphenicol acetyltransferase (CAT) gene, green fluorescent protein (gfp) gene (Haseloff et al, 1997), and luciferase gene, amongst others.

The present invention is applicable to any plant, in particular a monocotyledonous plants and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea pluriuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chaenomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Diheteropogon amplectens, Dioclea spp, Dolichos spp., Dorycnium rectum, Echinochloa pyramidalis, Ehrartia spp., Eleusine coracana, Eragrestis spp., Erythrina spp., Eucalyptus spp., Euclea schimperi, Eulalia villosa, Fagopyrum spp., Feijoa sellowiana, Fragaria spp., Flemingia spp, Freycinetia banksii, Geranium thunbergii, Ginkgo biloba, Glycine javanica, Gliricidia spp, Gossypium hirsutum, Grevillea spp., Guibourtia coleosperma, Hedysarum spp., Hemarthia altissima, Heteropogon contortus, Hordeum vulgare, Hyparrhenia rufa, Hypericum erectum, Hyperthelia dissoluta, Indigo incarnata, Iris spp., Leptarrhena pyrolifolia, Lespediza spp., Lettuca spp., Leucaena leucocephala, Loudetia simplex, Lotonus bainesii, Lotus spp., Macrotyloma axillare, Malus spp., Manihot esculenta, Medicago sativa, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativum, Podocarpus totara, Pogonarthria fleckii, Pogonarthria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium san guineum, Sciadopitys verticillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp. Vitis vinifera, Watsonia pyramidata, Zantedeschia aethiopica, Zea mays, amaranth, artichoke, asparagus, broccoli, brussel sprout, cabbage, canola, carrot, cauliflower, celery, collard greens, flax, kale, lentil, oilseed rape, okra, onion, potato, rice, soybean, straw, sugarbeet, sugar cane, sunflower, tomato, squash, and tea, amongst others, or the seeds of any plant specifically named above or a tissue, cell or organ culture of any of the above species.

Accordingly, the present invention clearly extends to any plant produced by the inventive method described herein, and any and all plant parts and propagules thereof. The present invention extends further to encompass the progeny derived from a primary transformed or transfected cell, tissue, organ or whole plant that has been produced by the inventive method, the only requirement being that said progeny exhibits the same genotypic and/or phenotypic characteristic(s) as that (those) characteristic(s) that has (have) been produced in the parent by the performance of the inventive method.

By “genotypic characteristic” is meant the composition of the genome and, more particularly, the introduced gene encoding the cell cycle control protein.

By “phenotypic characteristic” is meant one or more plant morphological characteristics and/or plant biochemical characteristics and/or plant physiological characteristics that are produced by ectopic expression of a cell cycle control protein in a plant.

Preferably, the plant is produced according to the inventive method is transfected or transformed with a genetic sequence, or amenable to the introduction of a protein, by any art-recognised means, such as microprojectile bombardment, microinjection, Agrobacterium-mediated transformation (including in planta transformation), protoplast fusion, or electroporation, amongst others.

The present invention is further described with reference to the following non-limiting Examples and to the drawings.

### EXAMPLE 1

### CELL CULTURE, PROTEIN AND ENZYME METHODS

Cell Culture

Suspension cultured cells of Nicotiana plumbiginifolia were grown in CS V medium supplemented with 9 μM 2,4-dichlorophenoxyacetic acid and 0.23 μM kinetin, and were brought to arrest at the cytokinin control point by the omission of kinetin from the culture medium. Arrest of cell cultures was confirmed by cell counting.

Antibodies

Polyclonal antibodies were raised in rabbits using the carboxy terminal amino acid sequence of the tobacco Cdc2 protein, designated as Cdc2a, as an immunogen. This peptide has the amino acid sequence KRITARNALEHEYFKDIGYVP (SEQ ID NO: 1) and has been demonstrated by complementation analyses in yeast to be a functional homologue of Cdc2. The Cdc2a peptide was synthesised chemically, purified by HPLC and conjugated to keyhole limpet haemocyanin. Antibodies were also prepared against a recombinant GST-Cdc25 catalytic core fusion protein, that had been synthesised in Escherichia coli.

Assay of Cdc2 and Cdc25 Activities

Both Cdc2 and Cdc25 enzyme activities were extracted from tobacco cells, by grinding the cells in liquid nitrogen. For Cdc2 extraction, NDE buffer containing 25 mM HEPES (pH 7.2) with protease and phosphatase inhibitors was used. For Cdc25 extraction, PDE buffer, containing 25 mM MOPS (pH 7.2), 100 mM NaCl, 10 mM DTT, 5 mM EDTA, 1mM EGTA, 1% NP-40, 50 mM NaF, 0.5 mM PMSF, 3 μg ml⁻¹ leupeptin, and 20 μg ml⁻¹ aprotinin, was used.

Immunoprecipitates of Cdc2 and Cdc25 were obtained by reaction with 25 I protein A-purified antibodies against Cdc2 and Cdc25 respectively, for 3 h at 4° C., followed by sedimentation of the antigen-antibody complexes using 35 μl protein A beads per sample. The immunoprecipitates were then washed three times, for 10 min per wash, using HDW buffer, followed by similar washing using HBK buffer. In the case of Cdc25 immunoprecipitates, the HBK buffer was supplemented with 2 μM spermidine.

To measure Cdc2 activity, the phosphorylation of H1 histone was followed.

To measure Cdc25 activity, assays were conducted in two stages. First, Cdc25 immunoprecipitates from 500 μg total soluble plant protein were incubated for 30 min at 30° C. in Cdc25 assay buffer with 0.25 μg tyrosine phosphorylated Cdc2 substrate that had been purified with p13^{suc1}-beads from 500 μg protein of arrested Cdc25-22 mutant fission yeast. The phosphatase reaction was stopped by removing the complexed Cdc25/Cdc2 by sedimentation. In the second stage of the Cdc25 assay, the supernatant was assayed for yeast Cdc2 kinase that had been activated. Assays to be compared directly were run and exposed together in a Phosphorimager.

Cdc2a Phosphotyrosine Assay

To assay phosphotyrosine in Cdc2a, the Cdc2a enzyme fraction was recovered essentially as described supra for the Cdc2 activity assay, except that 5 mg of extracted plant protein was used as starting material, and the NDE buffer was modified to include 2.5 mM sodium vanadate and 1 mM phosphotyrosine, and the immune complexes were washed with HDW buffer supplemented with I mM with sodium vanadate.

Western blots of cell-derived protein were probed with anti-phosphotyrosine mouse monoclonal (PY99, Santa Cruz Biotechnology, S.C., USA), followed by [¹²⁵I]-labelled second antibody, and the signal obtained was detected by Phosphorimage analysis.

Northern Blots

RNA was extracted from cells ground in liquid nitrogen, into 2 volumes of 10 mM Tris/HCl (pH 8.0), 100 mM NaCl, 1 mM EDTA, 1% (w/v) SDS and 2 volumes of phenol:chloroform:iso-amylalcohol 25:24:1 at 4° C. and fractionated.

RNA was electrophoresed on agarose gels, transferred to membrane and probed with the 65- bp BgIII-XbaI fragment of the Cdc25 gene, using standard procedures.

### EXAMPLE 2

### Expression of Yeast Cdc25 Makes Cell Division in Plant Cells Independent of Cell Cytokinin

The effect of ectopic expression of yeast Cdc25 in plant s was investigated because cells arrested by lack of cytokinin, whether derived from suspension culture or excised freshly from the plant, have abundant Cdc2 protein that is enzymically inactive because phosphorylated at tyrosine.

Latent Cdc2 protein kinase activity can be released in vitro by incubation with the phosphoprotein phosphatase Cdc25 that is specific for Cdc2. When cytokinin stimulates entry into mitosis, dephosphorylation of Cdc2 is one of the events that occur, but it was uncertain whether the hormone might have several effects in the cell cycle. To test this possibility we therefore arranged the inducible expression of the fission yeast Cdc25 gene in tobacco under the control of a dexamethasone-inducible promoter. Only if the sole essential action of cytokinin is to cause dephosphorylation and activation of Cdc2 kinase can the ectopic expression of the Cdc25 gene substitute for presence cytokinin at mitosis.

We now report that the sole essential action of cytokinin in sustaining cell division is activation of Cdc25 since the hormone can be substituted by expression of this gene.

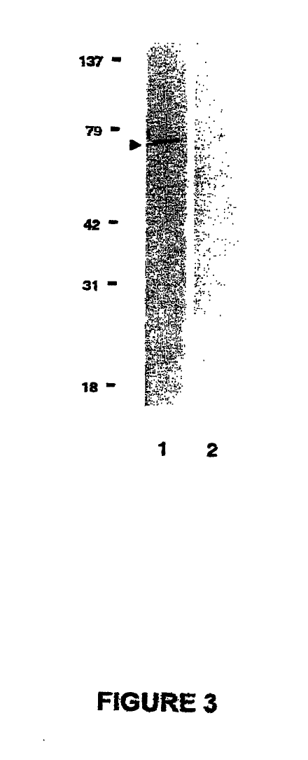

Levels of the fission yeast enzyme Cdc25 that removes inhibitory phosphate from tyrosine in Cdc2 kinase were brought under genetic control in the plant by joining the yeast Cdc25 gene (Russell and Nurse, 1986 incorporated herein by way of reference) to a modified plant promoter that contained rat glucocorticoid response elements (GREs), which are responsive to rat glucocorticoid receptor protein (GR) in the presence of dexamethasone (Schena et al., 1991 incorporated herein by way of reference) and therefore allowed induction without interference from plant hormones. The GRE-Cdc25, together with the constitutively-expressed NOS promoter-GR construct (Schena et al., 1991), were inserted into the well known and publicly available vector pBin19 (NCBI Accession No. U09365), which contains pnos:nptll for kanamycin resistance, using standard procedures (e.g. as described by Ausubel et al., Current Protocols in Molecular Biology, Wiley lnterscience, ISBN 047150338, 1992, which is herein incorporated by reference). This is done conveniently by cloning the entire Cdc25 coding region (SEQ ID NO: 2) into the BamHI/XbaI/SaII/PstII/SphII/HindIII polylinker immediately downstream of the modified GRE promoter described in FIG. 3 of Schena et al (1991) (SEQ ID NO: 4), and then sub-cloning the resultant GRE-Cdc25 gene construct into a suitable restriction site of pBin19 (SEQ ID NO: 5). For example, the Cdc25 coding region is readily inserted as a blunt-ended fragment into the blunt-ended BamHi site of the polylinker region in SEQ ID NO: 4, and recombiants with the fragments in the correct orientation identified by sequence analysis. The recombinant GRE-Cdc25 construct in pBinl9 was then introduced into protoplasts of N. plumbaginifolia by electroporation, and the transfected protoplasts used to establish cell lines. Clones resistant to kanamycin were tested for ability to form a colony on solid medium containing dexamethasone and auxin but no cytokinin.

At the high concentration of 10 μM dexamethasone, cells commonly arrested at prophase in mitotic catastrophe but lower inducer concentrations allowed colony formation and generated cell lines in which inducible expression of Cdc25 was detected by Western blot analysis using antibody against glutathione-S-transferase (GST)-Cdc25 fusion protein.

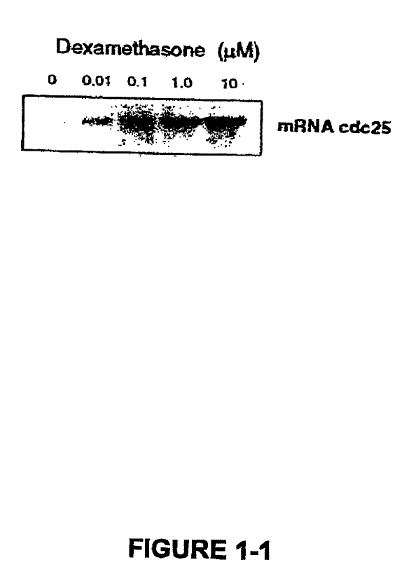

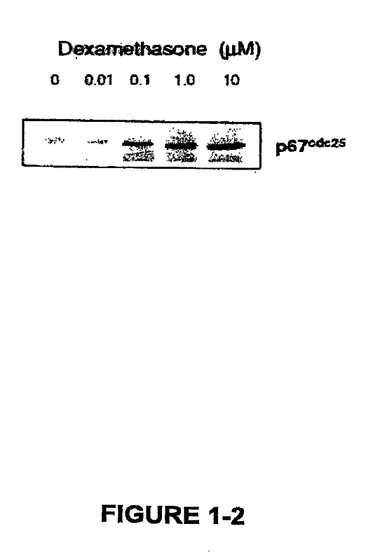

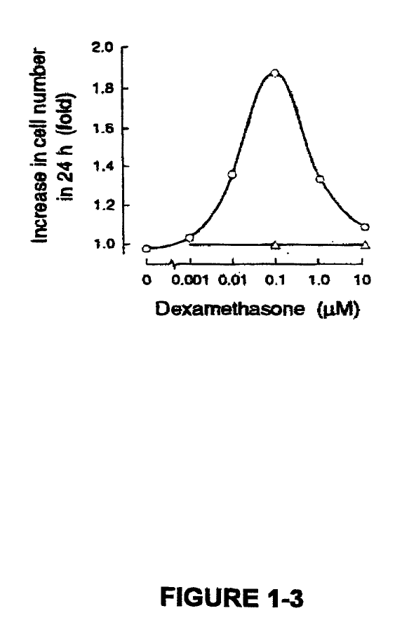

Inducible cell lines contained yeast Cdc25 DNA (detected in Southern blots, not shown) and in 0.01-10 μM dexamethasone they accumulated Cdc25 mRNA and protein (FIG. 1a; FIG. 1b). Effects on division were tested in cells that had been arrested at the G2 phase hormonal control point by depletion of auxin and cytokinin followed by provision of auxin only. Dexamethasone at 0.01-10 μM induced division (FIG. 1c) and a sharp optimum concentration of 0.1 μM dexamethasone was observed in independent clones, consistent with requirement for a critical optimum Cdc25 activity. No cell division was observed without inducer, or in untransformed cells treated with dexamethasone (FIG. 1c). Three independent lines were analysed biochemically and had similar properties. Results from one line are shown.

The experimental system used for subsequent experiments involved the prior arrest of suspension culture cells, at the cytokinin control point in late G2 phase. Arrest at this point was obtained by incubation without hormone and then with auxin (2,4-D) without cytokinin. Mitosis could then be induced by addition of cytokinin, or alternative potentially mitogenic treatments could be tested. Progress through prophase is a little slower after this arrest than in cells not emerging from hormonal block and is very suitable for study of the succession of biochemical events in plant mitosis.

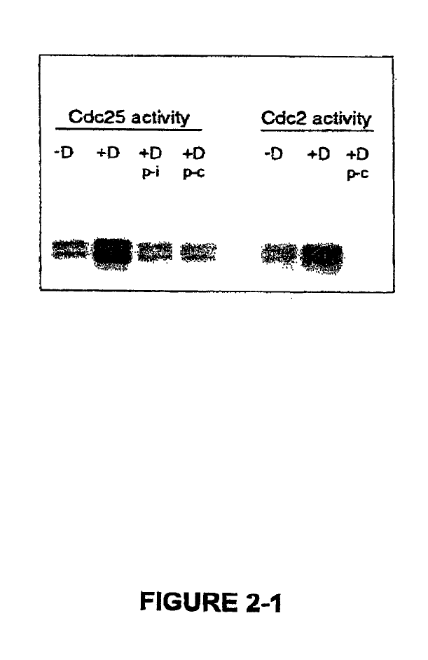

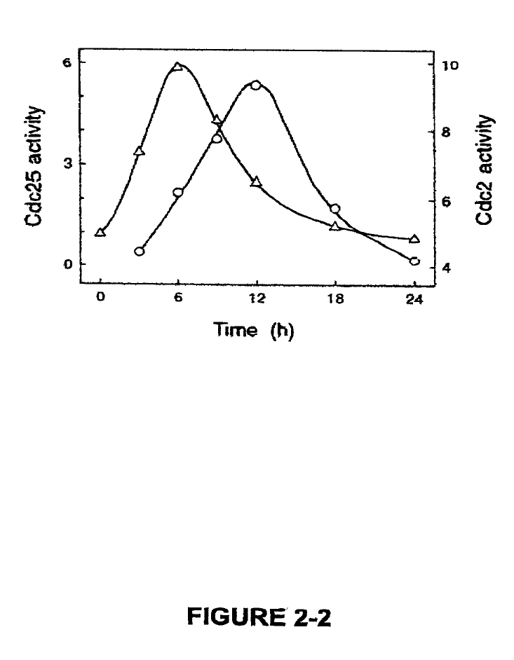

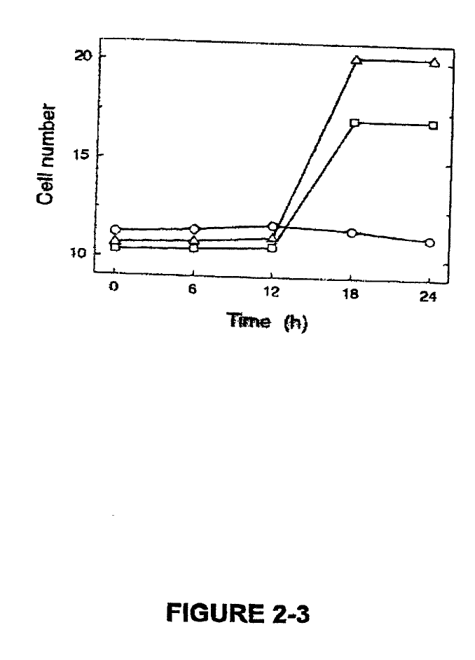

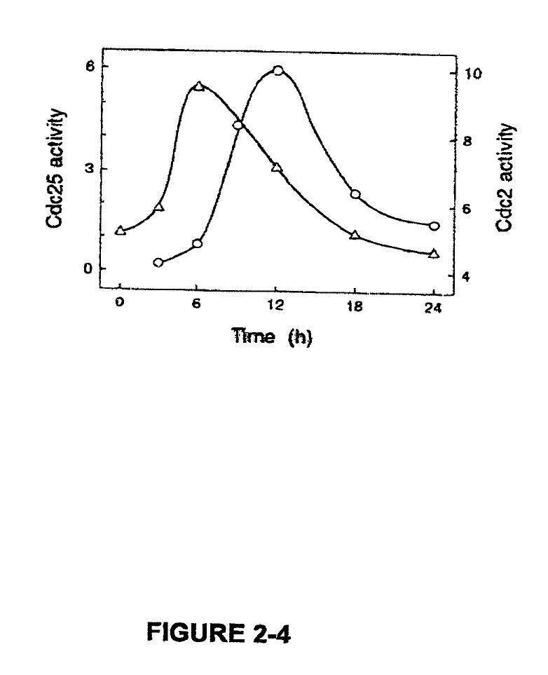

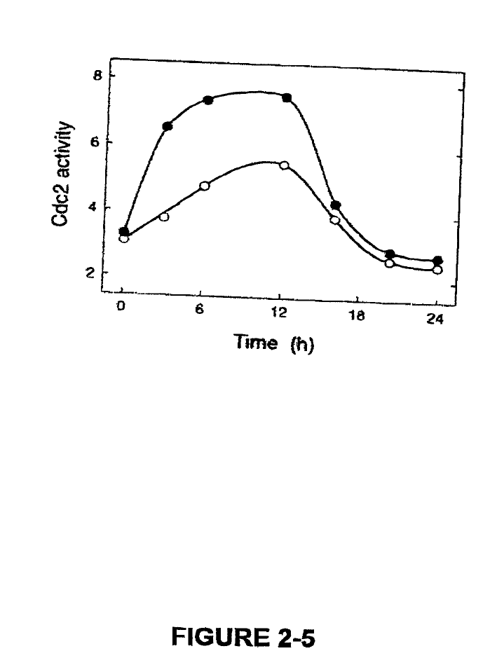

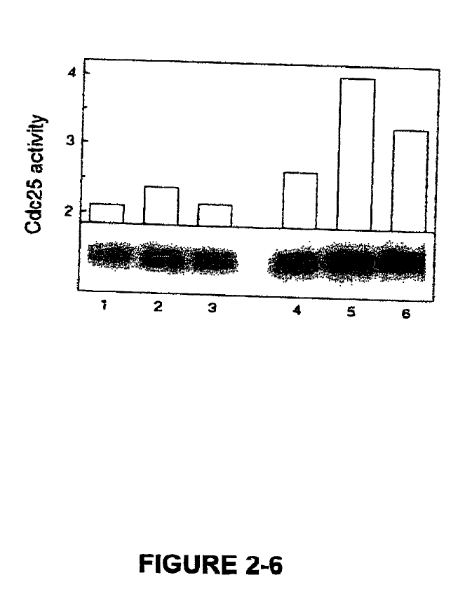

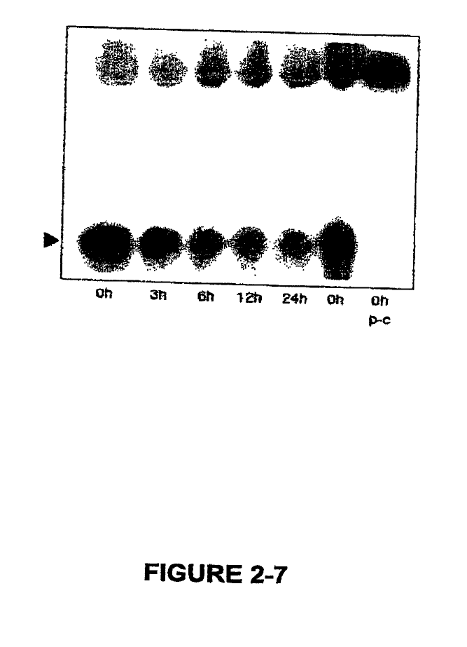

Induced synthesis of Cdc25 in cells at the cytokinin control point in late G2 resulted in appearance of Cdc25 activity, which was detected by its activation of yeast Cdc2 Hi histone kinase that was provided a substrate in low activity form, phosphorylated on tyrosine 15 and amenable to activation by Cdc25 (FIG. 2a to FIG. 2g). The induced Cdc25 phosphatase activity peaked at 6 h and provides an explanation for the increase in Cdc2 kinase activity, which increased while Cdc25 was active (FIG. 2b). Specific recovery of Cdc2a and Cdc25 was indicated by precompetition with Cdc2a peptide antigen and by preimmune anti-Cdc25 serum or anti-Cdc25 antibody precompeted with inactive GST-Cdc25 (FIG. 2a).

To test whether the effectiveness of ectopically expressed Cdc25 derived from the operation of mechanisms present in normal mitosis, transgenic cells induced with dexamethasone were monitored for Cdc25 phosphatase and Cdc2 kinase activity (FIG. 2b; FIG. 2c) in parallel with cells induced with cytokinin (FIG. 2c; FIG. 2d). Both showed increase in Cdc25 activity and then Cdc2a kinase activity leading to division. A control over Cdc25 activity at post-translational level is indicated by the absence of a higher Cdc25 catalytic activity when yeast enzyme was expressed in addition to the endogenous Cdc25 (FIG. 2b; FIG. 2d). This suggests that the additional yeast enzyme comes under homeostatic controls that are conserved between yeasts and plants. Post-translational control of Cdc25 activity is known to be complex, tolerant to different levels of the protein, and to involve activating phosphorylations and ubiquitin-directed proteolysis.

The temporal correlation of induced Cdc25 phosphatase activity with increase in Cdc2 kinase activity (FIG. 2b; FIG. 2d) suggested that the phosphatase is responsible. We tested this by investigating whether Cdc25 enzyme could activate Cdc2 from cells in prophase and whether the extent of activation by Cdc25 declined when activation had already occurred in vivo. Data presented in FIG. 2e show that excess bacterially-synthesised GST-Cdc25 could activate plant Cdc2 enzyme that was extracted in prophase between 3 hours and 12 hours, and that the extent of activation declined in proportion with activation that had previously occurred. These data are consistent with the increase in Gdc25 phosphatase driving prophase progression by dephosphorylating Cdc2. After 12 hours, Cdc2 activity declined during anaphase and the enzyme then became unresponsive to GST-Cdc25, consistent with the anaphase decline in activity being due to proteolysis of cyclin, as observed for cyclin 1b in maize mitosis.

The low level of Cdc25 activity in cells that are arrested by limiting cytokinin, as at time zero (i.e. 0 hours) in FIG. 2, indicates that down-regulation of Cdc25 activity is part of the cytokinin control mechanism and that induced Cdc25 therefore provides a biologically relevant signal. The resulting daughter cells were viable; indicating that mitosis driven by induced Cdc25 is functionally normal. These daughter cells could proliferate indefinitely with dexamethasone replacing cytokinin and required nine-fold dilution every 7 days precisely as in control cultures provided with auxin and cytokinin. They are routinely maintained in dexamethasone without cytokinin. Thus, unexpectedly the data provided herein reveal that the sole essential action of cytokinin in sustaining cell division is activation of Cdc25 and the hormone can be substituted by expression of the Cdc25 gene.

### EXAMPLE 3

### Evidence for the Presence of Cdc25 Protein in Plant Cells

The ability of yeast Cdc25 to influence cytokinin-mediated cell division in plants suggested to the present inventors that the yeast protein replaces the activity of an endogenous plant Cdc25 enzyme that is activated by cytokinin. To demonstrate that this is the case, the effectiveness of induced yeast Cdc25 produced in transformed plant cells to activate Cdc2, was compared to the effectiveness of a putative plant-derived Cdc25 from genetically unmodified cells to activate Cdc2.

The yeast and putative plant Cdc25 enzymes recovered by immunoprecipitation using anti-Cdc25 antibody were compared in reaction with tyrosine phosphorylated plant Cdc2 enzyme taken from cells arrested at the G2 control point (FIG. 2f, lanes 1-3). Substrate Cdc2 was also taken from cells after 3 hours stimulation with cytokinin (FIG. 2f, lanes 4-6), when partial Cdc2 activation had occurred (FIG. 2e; FIG. 2f, lanes 1 and 4).

The activation of plant Cdc2 by yeast Cdc25 expressed in plant cells (FIG. 2f, lane 6) demonstrates a mechanism by which Cdc25 can substitute for cytokinin. Furthermore this activation mechanism is a normal part of plant mitosis, because non-transgenic plant cells also contain a Cdc25 activity, unambiguously of plant origin, that is both present following cytokinin stimulation and capable of activating plant Cdc2 (FIG. 2f, compare lanes 2 and 5). Moreover, the plant Cdc25 activity is slightly more effective than the heterologous yeast Cdc25 in activating plant Cdc2 in the tobacco cells tested (FIG. 2f, compare lanes 5 and 6).

We also assayed phosphotyrosine in the Cdc2a kinase that increased in activity when the hormonal block was released. As shown in FIG. 2g, levels of tyrosine phosphate in Cdc2a declined after induction of Cdc25, as the catalytic activity of Cdc2a increased (FIG. 2b), indicating that a decline in phosphotyrosine caused by induction of Cdc25 in transgenic cells stimulates entry of cells into mitosis.

To further test the evidence for Cdc25 presence in genetically unmodified plant cells, we tested for immunological cross-reactivity between plant Cdc25 and authentic fission yeast Cdc25. In western blot analyses, antibodies against fission yeast Cdc25 detected a protein of 67 kDa in a tobacco cell fraction obtained using the mitotic protein p13^{suc1} as an affinity ligand to purify cell cycle proteins (FIG. 3, lane 1). Moreover, the binding of antibody to this 67 kDa tobacco protein was eliminated by pre-competition with authentic yeast Cdc25 protein (FIG. 3, lane 2), suggesting that the yeast and plant Cdc25 protein share protein epitopes, such as primary amino acid sequences, secondary, or tertiary structures. The size of the 67 kDa tobacco protein correlates with the known size of other Cdc25 molecules.

### EXAMPLE 4

### Expression of Cdc25 Under the Control of the Patatin Gene Promoter Increases Tuber Size and Number in Potato Plants

The fission yeast Cdc25 coding sequence is cloned between the promoter of a class I patatin gene ( Liu et al.., 1991) and the transcription termination signals of the nopaline synthase (NOS) gene of Agrobacterium fumefaciens. Preferentially, the B repeat region and the distal region of the A repeat of the patatin promoter is used, without the proximal region of the A repeat. The proximal region of the A repeat of the patatin promoter confers sucrose-responsiveness in various tissues, which is not a desirable characteristic for our purposes (Grierson et al., 1994). This construct is placed in a binary vector, mobilized to Agrobacterium tumefaciens, and the introduced into potato plants.

The Cdc25 protein is expressed under the control of the Class I patatin promoter when the first stolon starts to tuberize, consistent with the expression pattern for the patatin gene (Liu et al., 1991). At this stage, expression is associated with both internal and external phloem. After tuber induction has occurred, promoter activity is found both in tuberized stolons and in non-tuberized stolons. Expression then expands to the entire storage parenchyma, cortex and pith, but remains absent from the periderm.

Because the Class II patatin promoters are expressed in the periderm and as such are complementary to the Class I promoters (Köster-Töpfer et al.,; Liu et al., 1991; Nap et al., 1992), it is beneficial to have Cdc25 expression driven by both Class I and Class II promoters within the same plant. Because the Class I patatin promoter is not expressed before the first stolon initiates tuberization, no effects of Class I patatin-Cdc25 transgenes is seen on tuber initiation. However, the Class I patatin promoter drives Cdc25 expression very early after tuber initiation onwards, allowing a maximal impact of Cdc25 activity on organ formation and, as a consequence, on tuber size. The fact that the Class I patatin promoter activity subsequently also appears in non-tuberized stolons implies that the Class I patatin-Cdc25 transgene increases both the size and number of tubers.

### EXAMPLE 5

### Expression of Cdc25 Under the Control of the SAUR Gene Promoter or the A. rhizogenes rolB Promoter Increases Lignin in Poplar Plants

The fission yeast Cdc25 coding sequence is cloned between the promoter of the soybean SAUR gene (Li et al., 1992 ) and the transcription termination signals of the nopaline synthase (NOS) gene of Agrobacterium tumefaciens. The SAUR promoter is inducible by auxins. This chimeric gene construct is introduced between the T-DNA borders of the binary vector pBI121 or similar vector and mobilised into Agrobacterium tumefaciens. Poplar is transformed by Agrobacterium-mediated transformation using standard procedures.

Transgenic poplar trees containing this construct show increased lignin content, correlated with an increased stem diameter and the higher ratio of vascular tissue to pith and cortex cells.

A similar phenotype in poplar is produced when the Cdc25 expression is driven by the rolB promoter of Agrobacterium rhizogenes (Nilsson et al.,1997), that is expressed in cambial cells (i.e. the dividing cells of the vascular tissue).

### EXAMPLE 6

### Expression of Cdc25 Under the Control of Endosperm-specific Promoters Increases Grain Size and Yield of Grain Crop Plants

The fission yeast Cdc25 coding sequence is placed operably in connection with the endosperm-specific ltr1 promoter from barley, or a synthetic promoter containing the endosperm box (GCN motif) of the barley Hor2 gene (Vicente-Carbajosa et al., 1998). In each case, the Cdc25 structural gene is placed upstream of the transcription termination signals of the Agrobacterium tumefaciens nopaline synthase (NOS) gene. Cereals, in particular rice, maize, wheat and barley, are transformed using standard procedures, in particular microprojectile bombardment or Agrobacterium-mediated transformation systems, with the gene constructs.

The grain size and starch storage capacity of the endosperm of the seeds of transformed plants is increased relative to otherwise isogenic non-transformed plants.

### EXAMPLE 7

### Expression of Cdc25 Under the Control of Meristem-specific Promoters Reduces apical Dominance in A. thaliana and B. napus Plants

The fission yeast Cdc25 coding sequence is placed operably in connection with the shoot meristem-specific LEAFY promoter (Weigel et al., 1992), or the KNOTTED-like Arabidopsis thaliana knat1 promoter (Accession number AJ131822), or the KNOTTED-like Malus domestica kn1 promoter ( Accession No. Z71981), or the A. thaliana CLAVATA1 promoter (Accession number AF049870). In each case, the Cdc25 structural gene is placed upstream of the transcription termination signals of the Agrobacterium tumefaciens nopaline synthase (NOS) gene. A. thaliana and Brassica napus plants are transformed as described by Bechtold et al., 1993.

Transformed plants exhibit cytokinin-like effects at the level of the shoot (and flower) meristem, resulting in reduced apical dominance.

### EXAMPLE 8

### Expression of Cdc25 Under the Control of the cab-6 or ubi7 Promoters Reduces Leaf Necrosis and Chlorosis in Lettuce Plants

The fission yeast Cdc25 coding sequence is placed operably in connection with the leaf-specific cab-6 gene promoter derived from Pinus (Yamamoto et al., 1994) or senescence-specific ubi7 gene promoter (Garbarino et al., 1995). In each case, the Cdc25 structural gene is placed upstream of the transcription termination signals of the Agrobacterium tumefaciens nopaline synthase (NOS) gene. Lettuce is transformed as described by Bechtold et al., 1993.

Leaf deterioration (chlorosis and necrosis) in lettuce, for example as a consequence of post-harvest storage, is delayed in transformed lettuce plants compared to non-transformed control plants.

### EXAMPLE 9

### Expression of Yeast Cdc25 Under Growth Limiting Conditions Overrides the Block in DNA Replication

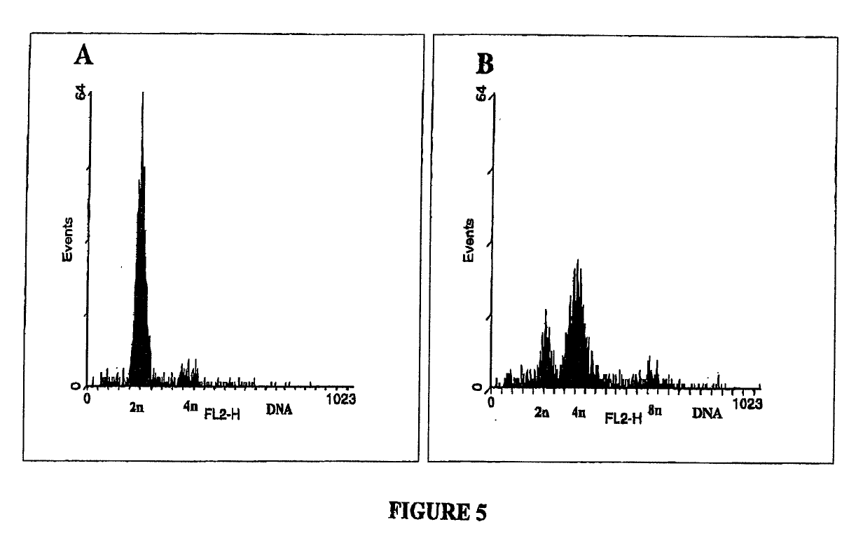

Methods: Cells of N. plumbaginifolia were maintained in CSV medium containing 9 millimolar 2,4-D and 0.23 millimolar kinetin and were diluted every seven days by transferring 5ml culture into 50 ml of fresh medium. Cells in which the full length Cdc25 gene could be inducibly expressed were created by joining the fission yeast Cdc25 gene to a modified plant promoter that contained rat glucocorticoid response elements (GREs), which are responsive to rat glucocorticoid receptor protein (GR). When inserted into the genome of the transgenic plant cells induction could be obtained by the presence of dexamethasone. The GRE-Cdc25, together with the constitutively-expressed NOS promoter-GR construct, were inserted into the vector pBinl9, which contains pnos:nptil for kanamycin resistance, and was introduced into cells of N. plumbaginifolia by electroporation into protoplasts. Clones resistant to kanamycin were selected and their arrest properties investigated. Non transgenic cells (A) and transgenic cells (B) were cultured in identical medium containing 0.1 micromolar dexamethasone (see FIG. 5). After 6 days of growth, when cell cycle progression was arrested in the non-transgenic cells, cells were harvested by centrifugation at gav=1643 for 3 min in a swing out head. Cells were washed once with washing buffer (0.3 M Mannitol, 0.12 M NaCl, 3.0 mM 2-(N-Morpholino)ethanesulfonic acid (MES), ph 5.8) then resuspended in two volumes of digestion solution (washing buffer supplemented with cell wall digesting enzymes; with 2% (w/v) cellusin (Calbiochem), 2% drisease (Fluka), 2% macerozyme (Calbiochem) and 4% hemicellulase (Sigma). Wall digestion was carried out at 37° C. with shaking at 120-125 rpm and was monitored by microscopy for 90 min until all cells were converted into spherical protoplasts. The protoplasts were collected by centrifugation at 5000 rpm (gav-42) for 3 min and washed once with washing buffer. Release of the nuclei was done by adding 2 ml of Galbraith buffer (45 mM Mg Cl₂, 30 mM sodium citrate, 20 mM 2-(N-Morpholino) propanesulfonic acid (MOPS), 1% Triton X-100, pH 7.0. Nuclear release was monitored by microscopy and was complete by 5 min, then debris was discarded by filtering through a 30 micrometer-pore Nylon filter twice. Nuclei were stained with propidium iodide (PI) by incubation in PI solution of 20 microgram/ml in 1 mM ethylenediaminetetraacetic acid (EDTA) in a 25° C. waterbath for 15 min. Then the nuclei were fixed by adding paraformaldehyde to a final concentration of 2% and incubated in a 25° C. waterbath for 15 min. Nuclear DNA content was examined by flow cytometry in a Becton Dickinson FACScan with filter wavelength for FL-2 of 585 nm. Data-analysis was done with WinMDI 2.7 written by Joseph Trotter, The Scripps Research Institute, La Jolla, Calif. 92037.

FIG. 5 indicates the frequency profile of nuclei with 2n, 4n and 8n amounts of DNA isolated from cultures in which cell division is arresting, in (A) nontransgenic cells, (B) transgenic cells in which the Cdc25 gene is joined to a glucocorticoid regulated promoter and was induced by the presence of 0.1 micromolar dexamethasone. Both cultures were sampled after 6 days of batch culture, when cell cycle progress has arrested at a G1/S control point in non-transgenic cells (2N peak in A) but has been driven through this point in a majority of the transgenic cells resulting in accumulation mostly in G2 phase (4n peak in B) and in some cases an additional traverse of S phase has been induced without intervening mitosis, resulting in endoreduplication of the genome and generation of 8n nuclei (seen in B).

References cited herein are incorporated in their entirety to the extent they are not inconsistent herewith.

### REFERENCES

1. An et al., EMBO J 4:277-284, 1985

2. An et al., Plant J. 10(1): 107-121, 1996

3. Albani, et al., Plant Mol. Biol. 15: 605, 1990.

4. Albani, et al., Plant Mol. Biol. 16: 501, 1991.

5. Albani et al., Plant Cell 9: 171-184, 1997

6. Arion et al., Cell 55:371-378, 1988.

7. Armstrong, et al.Plant Cell Reports 9: 335-339, 1990.

8. Arnoldo, et al., J. Cell. Biochem., Abstract No. YIOI, 204, 1992.

9. Baker et al., Plant Mol. Biol. 24: 701-13, 1997

10. Baltz, et al., The Plant J. 2: 713-721, 1992.

11. Barthels et al., The Plant Cell 9:2119-2134, 1997

12. Barros et al., Plant Mol. Biol. 19(4): 665-75, 1992

13. Baszczynski, et al., Nucl. Acid Res. 16: 4732, 1988.

14. Baszczynski, et al., Plant Mol. Biol. 14: 633, 1990.

15. Bechtold, N. J., et al., C.R. Acad. Sci. (Paris, Sciences de la vie/Life Sciences)316: 1194-1199, 1993.

16. Bell et al, Plant Mol. Biol. 23:445-451, 1993.

17. Bhattacharyya-Pakrasi, et al, The Plant J. 4: 71-79, 1992.

18. Binarova et al, Plant J 16:697-707, 1998

19. Bird and Wilson, Mol. Plant-Microbe Interact. 7:419-42, 1994

20. Bögre et al, Plant Physiol 113: 841-852, 1997

21. Bögre et al, Plant Cell 11: 101-114, 1999

22. Buchholz et al., Plant Mol. Biol. 25(5): 837-43, 1994

23. Calderini et al, J. Cell Sci. 111: 3091-3100, 1998

24. Cejudo, F. J., et al. Plant Molecular Biology 20:849-856, 1992.

25. Chaffield J M, Armstrong D J., Plant Physiol. 84: 726-731, 1987.

26. Chaudhary et al., Plant Mol. Biol. 30(6): 1247-57, 1996

27. Christensen et al., Plant Mol. Biol. 18:675-689, 1992

28. Chung and Parish, FEBS Lett. 3;362(2): 215-9, 1995

29. Cleveland, T. E. et al., Plant Mol. Biol. 8:199-208, 1987.

30. Colasanti et al.,Proc. Natl. Acad. Sci. USA 88:3377-3381, 1991.

31. Conkling, et al., Plant Physiol. 93:1203,1990.

31. Christou, P., et al. Plant Physiol 87: 671-674, 1988.

33. Cohen-Fix and Koshland, Curr. Opin. Cell Biol. 9:800-806, 1997.

34. Crossway et al., Mol. Gen. Genet 202:179-185, 1986.

35. Crowell, et al., Plant Mol. Biol. 18: 459-466, 1992.

36. Cummins, et al., Plant Mol. Biol. 19: 873-876, 1992

37. de Pater et al., Plant J. 2: 837-44, 1992

38. De Veylder et al., FEBS Lett 412:446-452, 1997

39. Doerner et al., Nature 380:520-523, 1996.

40. Dolferus et al, Plant Bio. Physiol. 105(4): 1075-87, 1994 Aug

41. Ebel, J., et al., Arch. Biochem. Biophys. 232:240-248,1984.

42. Ebert, et al., Proc. Nat Acad. Sci. 84: 5745-5749, 1987.

43. Elledge, Science 274 1664-1672,1996.

44. Ellis et al., EMBO Journal 6:11-16, 1987.

45. Ellis, et al., Plant Mol. Biol. 10:203-214, 1988.

46. Evans et al., Cell 33:389-396, 1983.

47. Fantes, P. Nature 279:428-430, 1979.

48. Fantes P. & Nurse P. In: The Cell Cycle (ed. John, P.) Cambridge University Press, 11-33,1981.

49. Fantes P., Curr. Opin. Cell Biol. 1: 250-255, 1989

50. Feiler H. S., Jacobs T., Proc. Nat Acad. Sci. 87:5397-5401, 1990.

51. Fenoll et al in Cellular and Molecular Aspects of Plant-nematode Interactions; Kluwer Academic C. Fenoll, FMW-Grundler and SA Ohl (Eds), 1997

52. Fesquet et al., EMBO J. 12:3111-3121, 1993.

53. Francis D. & Halford N. G. Physiol. Plant 93:365-374, 1995.

54. Francis, D., Dudits, D., and Inze, D. Plant Cell Division, Portland Press Research Monograph X, Portland Press, London, seewhole of contents, 1998.

55. Fromm et al. Proc. Natl. Acad. Sci. (USA) 82:5824-5828, 1985.

56. Garbarino et al., Plant Physiol. 109: 1371-1378, 1995.

57. Gatz et al., Curr. Opinion Biotech. 7: 168-172, 1996.

58. Gordon, et al., J. Exp. Bot 44: 1453-1465, 1993.

59. Grierson et al.., Plant J. 5:815-826, 1994

60. Grimes, et al., The Plant Cell 4:1561-1574, 1992.

61. Grosset J, et al., Plant Mol. Biol. 34:231-238, 1997.

62. Guerrero et al., Mol. Gen. Genet 224: 161-168, 1993

63. Hajela et al., Plant Physiol. 93: 1246-1252, 1990

64. Hanahan, D. J. Mol. Biol. 166, 557-560, 1983.

65. Hamilton, et al., Plant Mol. Biol. 18: 211-218, 1992.

66. Haseloff, J., et al., Proc. Natl Acad. Sci. USA 94: 2122-2127, 1997.

67. Hayles J., and Nurse P. J. Cell Sci. Suppl. 4:155-170, 1986

68. Hayles et al., EMBO J. 5: 3373-3379, 1986.

69. Hemerly et al., Plant Cell 5:1711-1723, 1993.

70. Herrera-Estella et a., Nature 303: 209-213, 1983a.

71. Herrera-Estella et al., EMBO J. 2: 987-995, 1983b.

72. Herrera-Estella et al. In: Plant Genetic Engineering, Cambridge University Press, N.Y., pp 63-93, 1985.

73. Herzog, et al., Morgan, Joint DPGRG and BPGRG Symposium—Aspects and Prospects of Plant Growth Regulators, Monograph 6:151-164, 1980.

74. Hirt et al., Proc. Natl. Acad. Sci. USA 88: 1636-1640, 1991

75. Hirt et al., Plant J. 4:61-69, 1993

76. Hochstrasser, Genes Dev. 12:901-907, 1998.

77. Howard et al., Planta 170:535-540, 1987.

78. Huntley et al., Plant Mol. Biol. 37:155-169, 1998.

79. Ito et al., Plant Mol. Biol. 24: 863-878, 1994

80. Ito et al., Plant J 11:983-992, 1997

81. John, P. C. L. In:The Cell Cycle, Cambridge University Press, Cambridge, UK, 1981.

82. John PCL et al., Plant Cell 1:1185-1193, 1989.

83. John PCL et al, In: Ormrod J. C., Francis, D. (eds) Molecular and Cell biology of the Plant Cell Cycle. pp. 9-34, Kluwer Academic Publishers, Dordrecht, Netherlands, 1993.

84. Joshee et al., Plant Cell Physiol. 39: 64-72, 1998

85. Kasunga et al., Nature Biotechnology Vol 17: 287-291, 1999

86. Kirch et al., Plant Mol. Biol. 33:897-909, 1997

87. Klimyuk and Jones, Plant J. 11: 1-14, 1997

88. Kosugi et al, Upstream sequences of rice proliferating cell nuclear antigen (PCNA) gene mediate expression of PCNA-GUS chimeric gene in meristems of transgenic tobacco plants, Nucleic Acids Research 19:1571-1576, 1991.

89. Kosugi S. and Ohashi Y, PCF1 and PCF2 specifically bind to cis elements in the rice proliferating cell nuclear antigen gene, Plant Cell 9:1607-1619, 1997.

90. Köster-Töpfer et al., Mol. Gen. Genet. 219: 390-396.

91. Krens, F.A., et al., Nature 296: 72-74, 1982.

92. Krek, Curr. Opin. Genet. Dev 8:36-42, 1998.

93. Kumagai A. and Dunphy W. G., Cell 64:904-914,1991.

94. Kuhlemeier et al., Ann. Rev. Plant Physiol., 38:221-257, 1987.

95. Labbe J-C et al, EMBO J. 8:3053-3058, 1989.

96. Lam, E. et al., The Plant Cell 2: 857-866, 1990.

97. Lake R. S. & Salzman N. P. Biochemistry 11:4817-4825,1972.

98. Lanahan, M. B., et al., Plant Cell 4:203-211, 1992.

99. Langan T. A. Meth. Cell. Biol. 19:127-142,1978.

100. Lawton et al., Plant Mol. Biol. 19:755-43, 1992

101. Lebel et al, Plant J. 16:223-33, 1998

102. Lee M. G. and Nurse P., Nature 327:31-35,1987.

103. Lepetit et al., Mol Gen Genet 231:276-285, 1992

104. Lee et al., Plant Physiology 85:327-330, 1987.

105. Li et al., Plant Physiol. 118:407-417, 1998

106. Li et al., Devel. Biol. 153: 386-395,1992.

107. Lisztwan et al., EMBO J. 18:368-383, 1998.

108. Liu et al., Plant Mol. Biol. 153:386-395, 1991.

109. Lundgren, et al., Cell 64: 1111-1122, 1991.

110. Lyndon R. F., In: Balls M. Billeff F.S. (eds) The Cell cycle in Development and Differentiation. pp. 167-183. Cambridge University Press, Cambridge, UK, 1973.

111. Manners et al., Plant Mol. Biol. 38: 1071-80, 1998

112. Marcotte W. R.; Bayley C. C. and Quatrano R. S., Nature 335:454-457, 1988.

113. Marrs et al., Dev Genet 14: 27-41, 1993

114. Matzke et al, Plant Mol. Biol., 14:323-32, 1990

115. McElroy et al., Plant Cell 2:163-171, 1990

116. Melchers et al, Plant J, 5:469-80, 1994

117. Mena et al, The Plant Journal 116(1):53-62, 1998

118. Minronov et al., Plant Cell 4: 509-522, 1999

119. Molina and Garcia-Olmedo, FEBS Lett 316:119-22, 1993

120. Morgan et al., In: Jackson, M. B. ed., British Plant Growth Regulator Group, Monograph 9:75-86, 1983.

121. Morris R O, et al., Aust. J. Plant Physiol. 20: 621-637, 1993.

122. Murray, A. and Kirschner, M. Science 246:614-621, 1989.

123. Nakase et al, Plant Mol. Biol. 33:513-522, 1997

124. Nap et al., Plant Mol. Biol. 20: 683-694, 1992

125. Nasmyth K. Curr Opin. Cell Biol. 5:166-179, 1993.

126. Nasrallah, et al., Proc. Natl. Acad. Sci. USA 85: 5551, 1988.

127. Niebel et al, Mol. Plant-Microbe Interact 8; 371-8, 1995

128. Niwa et al., DNA Res. 1: 213-221, 1994.

129. Nilsson et al., Physiol. Plant. 100:456-462, 1997

130. Norbury C. & Nurse P. Ann. Rev. Biochem. 61:441-470, 1992.

131. Nurse, P. Nature 344: 503-508,1990

132. Nurse P. and Bissett Y. Nature 292:558-560, 1981.

133. Odell et al., Nature 313:810-812, 1985

134. Olsen O-A, et al., Seed Science Res. 2:117-131,1992.

135. Olsen et al., Trends Plant Sci. 4: 253-257, 1999

136. Oppenheimer, et al., Gene 63: 87, 1988.

137. Oppermanetal., Science 263:221-23, 1994

138. Ormrod, J. C., and Francis, D. (1993) Molecular and Cell Biology of the Plant Cell Cycle, Kluwer Academic Publishers, Dordrecht, Netherlands.

139. Ouellet et al, FEBS Lett 423:324-328, 1998

140. Pathirana, et al., Plant Mol. Biol. 20: 437-450, 1992.

141. Paszkowski et al., EMBO J 3:2717-2722, 1984.

142. Pearson, et al., Plant Mol. Biol. 18: 235-245,1992.

143. Pines J., Biochem J. 308:697-711, 1995a.

144. Pines J., Nature 376:294-295,1995b

145. Plesse et al., in Plant Cell Division, (Portland Press Research Monograph X, D. Francis, D. Dudits and D. Inze, eds (London: Portland Press) 145-163, 1998.

146. Poon et al., EMBO J. 12:3113-3118, 1993.

147. Postma-Haarsma et al., Plant Mol. Biol. 39:257-71, 1999

148. Reed et al., Proc. Natl. Acad. Sci, USA 82:4055-4959,1985.

149. Raghothama et al., Plant Mol. Biol. 23: 1117-28, 1993

150. Regad et al., Mol. Gen Genet 248; 703-711, 1995

151. Reiss and Bryngelsson, Physiol Mol. Plant Pathol., 49: 331-341, 1996

152. Renaudin J. P., et al., Plant Mol Biol 32:1003-1018, 1996

153. Riou-Khamlichi et al, Science 283: 1541-1544

154. Russel P. & Nurse, P. Cell 45:145-153, 1986.

155. Russel P. & Nurse P. Cell 49:559-567, 1987.

156. Russel, P. & Nurse, P. Cell 49:569-576, 1987.

157. Russell D A, Fromm M E Plant Physiology (suppl) 89: 112, 1997.

158. Sanford, J. C., et al., Particulate Science and Technology 5: 27-37,1987.

159. Sato et al., Proc. Natl Acad Sci USA 93: 8117-8122, 1996

160. Sauter, M. et al., Plant J 7: 623-632, 1995.

161. Schneider et al., Plant Physiol. 113: 335-45, 1997

162. Schoffl et al., Mol. Gen Genet 217:246-53, 1989

163. Schena M., and Lloyd, A M, Proc. Natl. Acad. Sci, USA 88:10421-10425,1991.

164. Scofield, et al., J. Biol. Chem. 262: 12202, 1987.

165. Shaul et al., Proc Natl Acad Sci USA 93:4868-4872, 1996

166. Simon, et al., Plant Mol. Biol. 5:191,1985.

167. Soni R et al, Plant Cell 7:85-103, 1995

168. Sorrel D. et al., Plant Physiol 119:343-352, 1999

169. Stalberg, et al, Planta 199: 515-519, 1996.

170. Stotz and Long, Plant Mol. Biol. 41:601-614,1999

171. Strittmatter et al., Mol Plant-Microbe Interact 9:68-73, 1996

172. Sun Y et al, Proc Natl Acad Sci USA 96:4180-4185, 1999

173. Suzuki et al., Plant Mol. Biol. 21: 109-119,1993.

174. Skriver, K., et al. Proc. Natl. Acad. Sci. (USA) 88: 7266-7270, 1991.

175. Swenson et al, Cell 47:861-870,1986.

176. Takaiwa, et al., Mol. Gen. Genet 208: 15-22, 1986.

177. Takaiwa, et al., FEBS Letts. 221: 43-47, 1987.

178. Tingey, et al., EMBO J. 6:1, 1987.

179. Traas et al., Curr Opin. Plant Biol. 1:498-503, 1998

180. Trehin et al, Plant Mol. Biol. 35:667-672, 1997

181. Trick, et al., Plant Mol. Biol. 15: 203,1990.

182. Tucker et al., Plant Physiol. 113: 1303-1308, 1992.

183. Turner et al., Proc Natl Acad Sci USA 94(8):3866-71,1997

184. Twell et al., Mol. Gen Genet. 217:240-245, 1989

185. Twell et al., Sex. Plant Reprod. 6:217-224, 1993

186. Van der Meer, et al., Plant Mol. Biol. 15, 95-109, 1990.

187. Van der Zaal, et al., Plant Mol. Biol. 16, 983, 1991.

188. Vicente-Carbajosa et al., Plant J. 13: 629-640, 1998.

189. Vignutelli et al., Plant J., 14: 285-95, 1998

190. Walker et al., Proc. Nati. Acad. Sci. (USA) 84:6624-6628, 1987.

191. Wang H. and Crosby, W. L., Nature 386: 451-451, 1997

192. Ward et al., Plant Cell 3:1085-1094, 1991

193. Waters et al., J. Experimental Botany Vol 47(296):328-338, 1996

194. Weigel et al., Cell 69:843-859, 1992.

195. Wiegand, R. et al, Plant Mol. Biol. 7: 235-243, 1986.

196. Wilhelm and Thomashow, Plant Mol. Biol. 23(5):1073-7, 1993 Dec

197. Wilson C., et al., Physiol. Plant 102:532-538, 1999

198. Wong et al., Plant J 9:587-599, 1996

199. Wu et al., Plant Cell Physiology 39(8):885-889, 1998

200. Wu et al., J. Biochem 123:386, 1998

201. Xie et al., EMBO J. 15:4900-4908, 1996.

202. Yamamoto et al., Plant Cell Physiol. 35:773-778, 1994.

203. Yang, et al., The Plant J 3: 573-585, 1993

204. Zeng et al., Nature 395: 607, 1998.

205. Zhang et al, Planta 200:2-12, 1996.

206. Zhang et al, Cytokinin acts on cell division through Cdc25 phosphatase (in press).

207. Zhang et al., Plant Science 129(1): 81-89, Oct 28 1997

208. Zhou et al., Plant J. 12:921-930, 1997.

We claim:

1. A method of modifying a plant character selected from the group consisting of a morphological character, a biochemical character, and a physiological character comprising expressing in a particular cell, tissue or organ of a plant an isolated gene encoding a Cdc25 protein or a homologue, analogue or derivative thereof operably under the control of a regulatable promoter sequence that is operable in a plant or a cell, tissue or organ thereof subject to the proviso that the modified plant character is not an enhancement of lateral root production.

2. The method according to claim 1, wherein the Cdc25 protein is a Cdc25 protein of fission yeast.

3. The method according to claim 1, wherein the Cdc25 protein is a Cdc25 protein of a plant.

4. The method according to claim 3, wherein the plant is a dicotyledonous plant.

5. The method according to claim 4, wherein the dicotyledonous plant is tobacco.

6. The method according to claim 1, wherein the regulatable promoter is an inducible promoter sequence.

7. The method according to claim 6, wherein the inducible promoter sequence is a dexamethasone-inducible promoter sequence.

8. The method according to claim 7, wherein the dexamethasone-inducible promoter sequence comprises a glucocorticoid response element (GRE).

9. The method according to claim 1 wherein the modified plant character is selected from the group consisting of: enhanced strength, enhanced stem thickness, enhanced stability, and enhanced wind-resistance, and wherein the regulatable promoter sequence is at least operable in the stem of a plant or a cell, or tissue thereof.

10. The method according to claim 9, wherein the promoter sequence is selected from the group consisting of: (i) a rbcs-1A gene promoter sequence; (ii) a rbcs-3A gene promoter sequence; (iii) a AtPRP4 gene promoter sequence; (iv) a T. bacilliform virus gene promoter sequence; and (v) a sucrose-binding protein gene promoter sequence.

11. The method according to claim 1 wherein the modified plant character is selected from the group consisting of: enhanced tuber formation and enhanced tuber development, and wherein the regulatable promoter sequence is at least operable in the tuber of a plant or a cell, or tissue of said tuber.

12. The method according to claim 11, wherein the plant is potato.

13. The method according to claim 12 wherein the promoter sequence is a potato patatin gene promoter sequence.

14. The method according to claim 13, wherein the patatin gene promoter sequence is selected from the group consisting of: (i) a class I patatin gene promoter sequence; and (ii) a class 11 patatin gene promoter sequence.

15. The method according to claim 14, wherein the class I patatin gene promoter sequence has a reduced number of functional sucrose-responsive elements compared to the naturally-occurring class I patatin gene from which said promoter sequence was derived.

16. The method according to claim 15 wherein the number of functional sucrose-responsive elements is reduced by deletion of a proximal region of the A repeat in said class I patatin gene.

17. The method according to claim 1 wherein the modified plant character is modified lignin content, and wherein the regulatable promoter sequence is at least operable in the cambium or vasculature of a woody plant, or a cell, tissue or organ of said cambium or vasculature.

18. The method according to claim 17, wherein the regulatable promoter sequence is selected from the group consisting of: (i) a cinnamoyl alcohol dehydrogenase (CAD) gene promoter sequence; (ii) a laccase gene promoter sequence; (iii) a cellulose synthase gene promoter sequence; and (iv) a xyloglucan endotransglucosylase (XET) gene promoter sequence.

19. The method according to claim 17, wherein the regulatable promoter sequence is the auxin-inducible SAUR promoter sequence.

20. The method according to claim 17 wherein the regulatable promoter sequence is the rolB promoter sequence.

21. The method according to claim 17, wherein the woody plant is selected from the group consisting of: Eucalyptus spp.; Populus spp.; Quercus spp.; Acer spp.; Juglans spp.; Fagus spp.; Acacia spp.; and teak.

22. The method according to claim 1 wherein the modified plant character is selected from the group consisting of: (i) enhanced seed set; (ii) enhanced seed size; (iii) enhanced grain yield; and (iv) enhanced endoreduplication in the seed of the plant, and wherein the regulatable promoter sequence is at least operable in the seed of a plant or a cell, tissue or organ of said seed.

23. The method according to claim 22, wherein the regulatable promoter sequence is selected from the group consisting of: (i) a barley Amy32b gene promoter sequence; (ii) a Cathepsin -like gene promoter sequence; (iii) a wheat ADP-glucose pyrophosphorylase gene promoter sequence; (iv) a maize zein gene promoter sequence; (v) a rice glutelin gene promoter sequence; (vi) a legumin gene promoter sequence; (vii) a napA gene promoter sequence; (viii) a Brazil Nut albumin gene promoter sequence; (ix) a pea vicilin gene promoter sequence; (x) a sunflower oleosin gene promoter sequence; (xi) a barley Itrl gene promoter sequence; and (xii) a barley Hor2 gene promoter sequence.

24. The method according to claim 22, wherein the regulatable promoter sequence is operable in the endosperm of the seed.

25. The method according to claim 23, wherein the regulatable promoter sequence comprises a rice prolamin NRP33 promoter sequence.

26. The method according to claim 23, wherein the regulatable promoter sequence comprises a synthetic promoter that contains a rice REB gene promoter sequence.

27. The method according to claim 1 wherein the modified plant character is enhanced bushiness or reduced apical dominance of the plant, and wherein the regulatable promoter sequence is at least operable in the meristem of a plant or a meristem cell.

28. The method according to claim 27 wherein the meristem is a lateral meristem.

29. The method according to claim 27 wherein the meristem is an apical meristem.

30. The method according to claim 27 wherein the regulatable promoter sequence comprises a LEAFY gene promoter sequence.

31. The method according to claim 27 wherein the regulatable promoter sequence comprises a knat1 gene promoter sequence.

32. The method according to claim 27 wherein the regulatable promoter sequence comprises a kn1 gene promoter sequence.

33. The method according to claim 27 wherein the regulatable promoter sequence comprises a CLAVATAI gene promoter sequence.

34. The method according to claim 1 wherein the modified plant character is enhanced nitrogen fixing capacity of the plant or a nodule of said plant, and wherein the regulatable promoter sequence is at least operable in the nodule of a plant or a cell, or tissue of said nodule.

35. The method according to claim 34, wherein the regulatable promoter sequence is selected from the group consisting of: (i) a nif gene promoter sequence; (ii) a nifH gene promoter sequence; (iii) a ENOD gene promoter sequence; (iv) a PEPC gene promoter sequence; (v) a leghaemoglobin gene promoter sequence; and (vi) a hemoglobin gene promoter sequence.

36. The method according to claim 1 wherein the modified plant character is reduced or delayed chlorosis or necrosis of the green leaf tissue of the plant, and wherein the regulatable promoter sequence is at least operable in the leaf of a plant or a cell, or tissue of said leaf.

37. The method according to claim 36, wherein the promoter is selected from the group consisting of: (i) a SAM22 gene promoter sequence; (ii) a rbcs-IA gene promoter sequence; (iii) a rbcs-3A gene promoter sequence; (iv) a cab-6 gene promoter sequence; and (v) a ubi7 gene promoter sequence.

38. The method according to claim 1 wherein the modified plant character consists of a partial or complete inhibition of the arrest of DNA replication in a plant cell under conditions that would otherwise arrest DNA replication in the cell.

39. The method according to claim 40 wherein the modified plant character comprises enhanced endoreplication or enhanced endoreduplication.

40. The method according to claim 1, wherein the modified plant character is enhanced cell expansion.

41. The method according to claim 1, wherein the regulatable promoter sequence comprises a regulatable cell-specific promoter sequence.

42. The method according to claim 1, wherein the regulatable promoter sequence comprises a regulatable tissue-specific promoter sequence.

43. The method according to claim 42, wherein the tissue-specific promoter sequence is selected from the group consisting of:(i) a phloem-specific promoter sequence; (ii) a cell-wall-specific promoter sequence; (iii) a root cortex-specific promoter sequence; (iv) a root vasculature-specific promoter sequence; (v) a tapetum-specific promoter sequence; and (vi) a meristem-specific promoter sequence.

44. The method according to claim 1, wherein the regulatable promoter sequence comprises a regulatable organ-specific promoter sequence.

45. The method according to claim 44, wherein the regulatable promoter sequence is selected from the group consisting of: (i) an aleurone-specific promoter sequence; (ii) a flower-specific promoter sequence; (iii) a fruit-specific promoter sequence; (iv) a leaf-specific promoter sequence; (v) a nodule-specific promoter sequence; (vii) a pollen-specific promoter sequence; (viii) an anther-specific promoter sequence; (ix) a root-specific promoter sequence; (x) a seed-specific promoter sequence; (xi) an endosperm-specific promoter sequence; (xii) an embryo-specific promoter sequence; and (xiii) a stigma-specific promoter sequence.

46. The method according to claim 1, wherein the regulatable promoter sequence comprises a regulatable cell cycle-specific promoter sequence.

47. The method according to claim 46 wherein the regulatable cell cycle-specific promoter sequence comprises a cell cycle gene promoter sequence.

48. The method according to claim 1, wherein the isolated gene encoding Cdc25 protein or a homologue, analogue or derivative is expressed by a process comprising introducing a gene construct that comprises said gene operably in connection with the regulatable promoter sequence into a plant cell and culturing said plant cell under conditions sufficient for transcription and translation to occur.

49. The method according to claim 49, wherein culturing of the plant cell under conditions sufficient for transcription and translation to occur includes organogenesis or embryogenesis.

50. The method according to claim 49 wherein the organogenesis or embryogenesis includes regeneration of the plant cell into a whole plant.

51. A transformed plant produced by the method according to claim 50.

52. A plant part, propagule, or progeny, of the plant according to claim 51, wherein said plant part, propagule or progeny exhibits a modified plant character selected from the group consisting of a modified morphological character, a modified biochemical character, and a modified physiological character as a consequence of the ectopic expression of Cdc25, or a homologue, analogue or derivative of Cdc25 therein.

53. A gene construct comprising an isolated gene encoding a Cdc25 protein or a homologue, analogue or derivative of Cdc25, placed operably in connection with a regulatable promoter sequence that is operable in a plant or a cell, tissue or organ of said plant, wherein said regulatable promoter sequence is selected from the group consisting of: (i) a dexamethasone-inducible promoter sequence; (ii) a patatin gene promoter sequence; (iii) a modified patatin gene promoter sequence having a deletion in a sucrose-responsive element; (iv) an auxin-inducible SAUR gene promoter sequence; (v) a rolB gene promoter sequence; (vi) a rice prolamin NRP33 gene promoter sequence; (vii) a synthetic promoter sequence comprising an endosperm box motif of the barley Hor2 gene; (viii) a LEAFY gene promoter sequence; (ix) a knat1 gene promoter sequence; (x) a kn1 gene promoter sequence; (xi) a CLAVATA1 gene promoter sequence; (xii) a cab-6 gene promoter sequence; (xiii) a rice REB gene promoter sequence; and (xiv) a ubi7 gene promoter sequence.

54. A transformed plant comprising the gene construct according to claim 53, wherein said plant exhibits a modified plant character compared to otherwise isogenic non-transformed plants selected from the group consisting of: (i) enhanced stem strength; (ii) enhanced stem thickness; (iii) enhanced stem stability; (iv) enhanced wind-resistance of the stem; (v) enhanced tuber formation; (vi) enhanced tuber development; (vii) increased lignin content; (viii) enhanced seed set; (ix) enhanced seed production; (x) enhanced seed size; (xi) enhanced grain yield; (xii) enhanced ploidy of the seed; (xiii) enhanced endosperm size; (xiv) reduced apical dominance; (xv) increased bushiness; (xvi) enhanced nitrogen-fixing capability; (xvii) enhanced nodulation or nodule size; (xviii) reduced or delayed leaf chlorosis; (xix) reduced or delayed leaf necrosis; (xx) partial or complete inhibition of the arrest of DNA replication in a plant cell under conditions that would otherwise arrest DNA replication in the cell; (xxi) enhanced endoreplication and/or enhanced endoreduplication; and (xxii) enhanced cell expansion.

55. A plant part, propagule, or progeny, of the plant according to claim 54, wherein said plant part, propagule or progeny exhibits the modified plant character as a consequence of the ectopic expression of Cdc25, or a homologue, analogue or derivative of Cdc25 therein.

56. The method according to claim 1 wherein the modified plant morphological and/or biochemical and/or physiological characteristic comprises an extended photosynthetic canopy of a crop plant, and wherein the regulatable promoter sequence is at least operable in the internode meristem of stem tissue of said crop plant.

57. The method according to claim 56, wherein the regulatable promoter sequence is a Proliferating Cell Nuclear Antigen (PCNA) promoter of rice.

58. The method according to claim 56 wherein the plant further exhibits enhanced grain yield.


SEQUENCE LISTING (5 sequences)

SEQ ID NO: 1 - protein, 21 aa, Artificial
  carboxy terminal amino acid sequence of the tobacco Cdc2 protein, designated as Cdc2a
KRITARNALEHEYFKDIGYVP

SEQ ID NO: 2 - DNA, 2745 bp, Schizosaccharomyces pombe
  CDS (491)..(2233): Fission yeast Cdc25
ttttcatattaatcaactgctttcttttaactttcttttttctttcattttttctttcaacgacagtatcttttatattctatattatagtcgtgaccgttggtgctcatctatttttgtctcattaatctctttttttttatatacttttattcggtcttcccgtcgtgtggagtctaaacttacttaaacttctttccggtttcttttgtaaaggttggttttgtttaatatttggctatattgctttcatcctgtctttcttttagttttctttttgtctcactttggcattaatatcttaaaccggtcttttgactcttccctaggattttcctttataatttatcttacttgctctattgatttctcattctgaaactcaatcttttgcagtcgtgtcgtcccattagttctttttgcagtgtacttggtttaaattaaatttaccattttgtctgcttttaataatagttaaacctcaactaaaatggattctccgctttcttcactttcctttaccaacactctatctggcaaacggaatgtattgcgtcctgctgcccgtgaattaaagttgatgtcggatcgcaatgcaaatcaagagctggattttttcttcccgaaatccaaacacattgcctcaacattagtggatccttttggaaaaacttgttcgacagcttcacctgcatcttccttagctgctgatatgtcaatgaacatgcatatcgatgaaagccctgccttaccgactcctcgtcgtacgctctttcgatctctttcttgtactgtagaaacccctctcgctaacaagactattgtttcacctctcccggagtcaccctcgaatgacgctttaaccgagtcctacttcttccggcaacctgcatcgaagtattccattacccaagattccccacgtgtttccagcactattgcttacagctttaagcctaaagcatcaatcgcattaaacaccaccaaaagcgaagctactcgttcgtcattatcgagttcttcttttgattcctaccttcgtcctaacgtctcacgttctcgatcatcaggcaacgcacccccatttttgcgatccagatcgagttcctcatattccattaacaaaaagaagggaacttctggtggacaagctacccgccatttgacttatgccttatcccgtacctgtagtcagtcgagcaacacgaccagcttacttgaaagttgtcttactgatgatacggatgatttcgaattaatgtctgatcatgaggatacatttacgatgggcaaggttgctgatttaccagaaagctctgttgaacttgttgaagacgctgcctctattcaacgtcccaacagtgattttggtgcttgcaatgataattctttagatgatctttttcaagcttcacccattaaacctattgatatgttacccaagataaataaagatattgcctttcctagcttgaaagttaggtccccttctccgatggcattcgctatgcaagaagatgcggaatatgatgagcaagatacaccagtcgttcgtcgtacccaaagcatgtttctcaattccacaagactagggcttttcaaaagccaagatcttgtgtgcgttacgccaaaacaatcgaccaaagaaagtgagcgcttcatctcttctcatgtcgaggatttatctcttccttgcttcgccgtgaaggaggactcattgaaacgaattacccaagaaacattgctcggtctattagatggtaaatttaaggatatttttgacaaatgcatcattattgattgccgttttgaatatgaataccttgggggccatattagcactgctgtgaatttaaatacgaaacaagctattgtggatgcctttttaagtaaacctcttactcatcgggttgcacttgtttttcattgtgaacatagtgcgcacagagcacctcatttggcattgcactttcgaaataccgacagacgaatgaatagtcatcgttatcctttcctttactatcccgaggtttatatacttcatggtggttacaagtcgttttacgaaaaccacaaaaaccgttgtgacccaattaactacgttccgatgaacgatcgttcgcatgttatgacctgcaccaaggctatgaataatttcaaacgaaacgctacttttatgcgtactaaaagttatactttttggccaaagtgtgttagcttccccagacgttaatgattctcctactgccatgcattccctctctacacttagaagattttaatgattttaggctgactcaatactttacctagcctgaagtatactcttatatattcaactttctaacctaagttttttctataaactttgaatcatgaaaccttttatttctattttttattattattattgaaagaaagcaatataccttttaaaaggtctcgacaacaaaaaatttaaactggctaaacttttcgaagtacatcacttttacgactaaatatctagaacgactagtttggttattattattacgcatggattttggtttaataattgattattttataatcaattgagcttaataatttctaagcaatcacgggtaaacatacgtaccgaagtccattctaattgctcttctcgcaattgctgttgataggaatgctaagaatttagcattctttgattgaagcagtaatgcgataggaactgagttgcttacgtttttgtaactctacctc

SEQ ID NO: 3 - protein, 580 aa, Schizosaccharomyces pombe
MDSPLSSLSFTNTLSGKRNVLRPAARELKLMSDRNANQELDFFFPKSKHIASTLVDPFGKTCSTASPASSLAADMSMNMHIDESPALPTPRRTLFRSLSCTVETPLANKTIVSPLPESPSNDALTESYFFRQPASKYSITQDSPRVSSTIAYSFKPKASIALNTTKSEATRSSLSSSSFDSYLRPNVSRSRSSGNAPPFLRSRSSSSYSINKKKGTSGGQATRHLTYALSRTCSQSSNTTSLLESCLTDDTDDFELMSDHEDTFTMGKVADLPESSVELVEDAASIQRPNSDFGACNDNSLDDLFQASPIKPIDMLPKINKDIAFPSLKVRSPSPMAFAMQEDAEYDEQDTPVVRRTQSMFLNSTRLGLFKSQDLVCVTPKQSTKESERFISSHVEDLSLPCFAVKEDSLKRITQETLLGLLDGKFKDIFDKCIIIDCRFEYEYLGGHISTAVNLNTKQAIVDAFLSKPLTHRVALVFHCEHSAHRAPHLALHFRNTDRRMNSHRYPFLYYPEVYILHGGYKSFYENHKNRCDPINYVPMNDRSHVMTCTKAMNNFKRNATFMRTKSYTFWPKCVSFPRR

SEQ ID NO: 4 - DNA, 235 bp, Artificial
  GRE-CaMV minimal promoter
gaattcgagctcggtaccgggccccccctcgactagagtcgactgtacaggatgttctagctactcgagtagctagaacatcctgtacagtcgagtagctagaacatcctgtacagtcgagtagctagaacatcctgtacagtcgagtagctagaacatcctgtacagtcgagtagctagaacatcctgtacagtcgactctaggcccgcaagacccttcctctatataaggaag

SEQ ID NO: 5 - DNA, 11777 bp, Artificial
  Cloning vector pBin19
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca